(19) United States
(12) Patent Application Publication
(10) Pub. No.: US 2025/0241974 A1
(43) Pub. Date: Jul. 31, 2025

(54) COMPOSITIONS AND METHODS FOR WOUND HEALING

(71) Applicant: University of Maine System Board of Trustees, Orono, ME (US)

(72) Inventors: Dorothy Klimis-Zacas, Orono, ME (US); Michael D. Mason, Orono, ME (US); Tolu Esther Alaba, Orono, ME (US); Panagiotis Tsakiroglou, Orono, ME (US); Blake Hamilton Turner, Orono, ME (US); Natalie Esther Vandenakker, Orono, ME (US); Jacob Allan Holbrook, Orono, ME (US)

(73) Assignee: University of Maine System Board of Trustees, Orono, ME (US)

(21) Appl. No.: 19/034,918

(22) Filed: Jan. 23, 2025

Related U.S. Application Data
(60) Provisional application No. 63/625,586, filed on Jan. 26, 2024.

(51) Int. Cl. A61K 36/45; A61K 9/06; A61K 31/192; A61K 31/216; A61K 47/10; A61K 47/12; A61K 47/36; A61P 17/02

(52) U.S. Cl. CPC A61K 36/45; A61K 9/06; A61K 31/192; A61K 31/216; A61K 47/10; A61K 47/12; A61K 47/36; A61P 17/02

(57) ABSTRACT

Materials and methods for modulating wound healing are described. Compositions including a phenolic acid extract of berries from the Vaccinium family are useful for promoting wound healing, and can be formulated in a cream, a serum or gel, or in a nanocellulose hydrogel.

### RELATED APPLICATIONS

This application claims priority to U.S. Provisional Application No. 63/625,586, filed under 35 U.S.C. § 111 (b) on Jan. 26, 2024, the entire disclosure of which is incorporated herein by reference for all purposes.

### BACKGROUND

Billions of dollars are spent annually on wound care in the United States, a lot of which is due to chronic wounds such as pressure and diabetic ulcers resulting from deficient vascular remodeling necessary for healing. There remains an unmet need for treatments for chronic wounds such as diabetic foot ulcers, ischemic limbs, and pressure ulcers. It would be advantageous to develop new compositions and methods for promoting wound healing.

### SUMMARY

Provided is a method of promoting wound healing in a subject, the method comprising administering an effective amount of a composition to a subject in need of wound healing to promote wound healing in the subject, wherein the composition comprises a nanocellulose hydrogel and an extract of berries from the Vaccinium family.

In certain embodiments, the berries are blueberries. In certain embodiments, the extract is a phenolic-rich fraction. In particular embodiments, the phenolic-rich fraction comprises chlorogenic acid. In particular embodiments, the phenolic-rich fraction further comprises ferulic acid and caffeic acid.

In certain embodiments, the nanocellulose hydrogel comprises a TEMPO-oxidized CNF hydrogel. In particular embodiments, the TEMPO-oxidized CNF hydrogel is crosslinked with citric acid.

Further provided is a composition comprising an extract of berries from the Vaccinium family; and a nanocellulose hydrogel. In certain embodiments, the extract comprises phenolic acids. In certain embodiments, the phenolic acids include chlorogenic acid. In particular embodiments, the phenolic acids further include ferulic acid and caffeic acid. In certain embodiments, the nanocellulose hydrogel comprises a TEMPO-oxidized CNF hydrogel. In particular embodiments, the TEMPO-oxidized CNF hydrogel is crosslinked with citric acid.

Further provided is a composition comprising a phenolic acid extract of berries from the Vaccinium family; phenethyl alcohol; hydrolyzed hyaluronic acid; and sodium hyaluronate; wherein the composition is in the form of a serum or gel. In certain embodiments, the phenolic acid extract comprises chlorogenic acid, ferulic acid, and caffeic acid.

Further provided is a method of promoting collagen remodeling in a wound, the method comprising administering to a wound an effective amount of a phenolic acid extract of berries from the Vaccinium family to promote collagen remodeling in the wound. In certain embodiments, the phenolic acid extract comprises chlorogenic acid, ferulic acid, and caffeic acid.

Further provided is a composition comprising a phenolic acid extract of berries from the Vaccinium family; shea butter glycerides; phenethyl alcohol; castor seed oils; cetearyl alcohol; glyceryl stearate; sodium stearoyl lactylate; balsam peru oil; wherein the composition is in the form of a cream. In certain embodiments, the phenolic acid extract comprises chlorogenic acid, ferulic acid, and caffeic acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: HPLC profile analysis of a non-limiting example PA fraction of wild blueberry (V. angustifolium) powder.

FIG. 2: Illustration showing the experimental design in Example I herein.

FIGS. 3A-3B: Treatment groups (FIG. 3A) and experimental timeline (FIG. 3B) used in Example I.

FIGS. 4A-4G: Images showing collagen remodeling results from Example I. Collagen remodeling scores are as defined in Table 1 in Example I.

FIG. 5 illustrates the experimental design used in Example II herein.

FIGS. 6A-6B: Treatment groups (FIG. 6A) and experimental timeline (FIG. 6B) used in Example II.

FIG. 7: Percentage wound closure across serum/gel (right) and cream (left) treatment groups in comparison to control untreated group.

FIG. 8: Endothelial cell count under microscope at 20× objectives across serum/gel treatment groups in comparison to control untreated group.

FIGS. 9A-9D: Vascularization score of representative wound area per group at 20× objectives annotated with the arrows. FIG. 9A shows mild vascularization of a wound in the control group. FIG. 9B shows moderate vascularization in the serum/gel control group. FIG. 9C shows high vascularization in the serum/gel 500 group. FIG. 9D shows mild vascularization in the serum/gel 1000 group.

FIGS. 10A-10D: Collagen remodeling score of representative wound area per group at 40× objectives annotated with the arrows. FIG. 10A shows mild deposition of collagen in the control group. FIG. 10B shows moderate deposition of collagen in the serum/gel control group. FIG. 10C shows dense or high deposition of collagen in the serum/gel 500 group. FIG. 10D shows mild deposition of collagen in the serum/gel 1000 group.

FIG. 11: Vascular protein expression qualified by immunoblotting analysis of VEGFA (left) normalized with beta-actin (right) across serum/gel groups compared with control group.

FIG. 12: Collagen protein expression qualified by immunoblotting analysis of Col1a1 (left) normalized with beta-actin (right) across serum/gel groups compared with control group.

FIGS. 13A-13B: Fold change expression of VEGFA (FIG. 13A) and Col1a1 (FIG. 13B) relative to housekeeping protein beta-actin, across serum/gel treatment groups in comparison with control untreated group.

FIGS. 14A-14D: Re-epithelialization score of epidermal layer of the skin representative wound area per group at 5× objectives annotated with the arrows. FIG. 14A shows no epithelialization in the control group. FIG. 14B shows no epithelialization in the serum/gel control group. FIG. 14C shows complete epithelialization in the serum/gel 500 group. FIG. 14D shows discrete incomplete epithelialization in the serum/gel 1000 group.

FIG. 15: Re-epithelialization protein expression by immunoblotting analysis of fibroblast growth factor (FGF7) (left) normalized with beta-actin (right) across serum/gel groups compared with control group.

FIG. 16: Re-epithelialization protein expression by immunoblotting analysis of epidermal growth factor receptor (EGFR) (left) normalized with beta-actin (right) across serum/gel groups compared with control group.

FIG. 17: Fold change expression of re-epithelialization proteins FGF7 and EGFR relative to house keeping protein beta-actin, across serum/gel treatment groups in comparison to the control untreated group.

FIG. 18: Illustration of TEMPO-oxidized CNF hydrogel preparation as described in Example III herein.

FIG. 19: Coomassie brilliant blue diffusion through TEMPO-oxidized CNF as varying concentrations.

FIG. 20: Photograph showing a spectrophotometer with a cuvette (light blue square) in the center.

FIG. 21: Absorbance value vs the concentration of CBB solution using the 600 μm optical cable and 1 second integration time.

FIG. 22: Results of a wound healing assay showing that a TEMPO-oxidized CNF hydrogel with a phenolic acid extract from wild blueberries promoted wound healing better than a control (nothing on the wound), a nanogel control (CNF without the extract), and Neosporin.

### DETAILED DESCRIPTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. All references cited herein, including books, journal articles, published U.S. or foreign patent applications, issued U.S. or foreign patents, and any other references, are each incorporated by reference in their entireties, including all data, tables, figures, and text presented in the cited references.

All publications mentioned in this description, are hereby incorporated in their entirety as if fully set forth herein to form part of the description.

Numerical ranges, measurements and parameters used to characterize the invention—for example, angular degrees, quantities of ingredients, polymer molecular weights, reaction conditions (pH, temperatures, charge levels, etc.), physical dimensions and so forth—are necessarily approximations; and, while reported as precisely as possible, they inherently contain imprecision derived from their respective measurements. Consequently, all numbers expressing ranges of magnitudes as used in the specification and claims are to be understood as being modified in all instances by the term “about.” All numerical ranges are understood to include all possible incremental sub-ranges within the outer boundaries of the range. Thus, a range of 30 to 90 units discloses, for example, 35 to 50 units, 45 to 85 units, and 40 to 80 units, etc. Unless otherwise defined, percentages are wt/wt %.

The present disclosure relates generally to compositions and methods for enhancing, promoting, stimulating, or improving wound healing. The compositions include specific extracts of berries from the Vaccinium family, such as blueberries. “Blueberry” is a general name for blue-colored berries of the Vaccinium family, but a number of different species may qualify as “blueberries” for purposes of this disclosure. Specifically, bilberries (European Blueberries or Vaccinium myrtillus) and the so-called “American” blueberries (various plants in the Vaccinium family) are all encompassed. The lowbush blueberry (Vaccinium angustifolium) has been ranked as one of the richest food sources of bioactive compounds (polyphenols) such as anthocyanins (ACNs) and phenolic acids (PAs) generally found in fruits and vegetables. The antioxidant activity of wild blueberries is a result of anthocyanins, procyanidins, chlorogenic acid, and other phenolic compounds. ACNs from wild blueberries are primarily composed of delphinidin, malvidin, petunidin, cyanidin, and peonidin. Their phenolic content is often influenced by growing practices, location, and harvesting methods. Scientists have documented that a person on average can consume 180-215 mg/day of ACNs with concentrations in the plasma in the pmol/L-nmol/L range.

Previously, it has been demonstrated that phenolic acid extract from wild blueberries significantly increased angiogenesis in HUVEC cells. However, the in vivo effects necessary for clinical translation were unknown. As previously described in U.S. Patent Application Publication No. 2020/0330541 A1, which is incorporated herein by reference, extracts of berries from the Vaccinium family are capable of inhibiting or increasing endothelial cell migration, angiogenesis, and/or gene expression of eNOS, RAC1, RHOA, VEGF, and AKT1. In accordance with the present disclosure, phenolic acid extracts of berries from the Vaccinium family, such as the phenolic-rich fractions described in U.S. Patent Application Publication No. 2020/0330541 A1, are useful for promoting wound healing in compositions formulated as a serum or gel, as a cream, or in a nanocellulose hydrogel.

In general, the compositions herein include a berry extract. The berry extract may be, for example, a phenolic acid extract such as the phenolic fraction described in U.S. Patent Application Publication 2020/0330541 A1, incorporated herein by reference. However, other extracts, and other phenolic acid extracts, are possible and encompassed within the scope of the present disclosure. In fact, it is not strictly necessary that the berry extract be a literal extract from berries. Rather, the berry extract may be a mixture of phenolic acids produced through synthetic or other means, such as a mixture of chlorogenic acid, caffeic acid, and ferulic acid.

In some non-limiting examples, the phenolic acid extract includes mainly chlorogenic acid along with caffeic acid and ferulic acid, and is a fraction soluble in, and extracted from blueberries with, ethyl acetate. Example protocols followed for the extraction of the bioactive phenolic compounds are described in detail by in Del Bo' C, Cao Y, Roursgaard M, Riso P, Porrini M, Loft S, et al., Anthocyanins and phenolic acids from a wild blueberry (Vaccinium angustifolium) powder counteract lipid accumulation in THP-1-derived macrophages, Eur J Nutr 2015, doi: 10.1007/s00394-015-0835-z, which is incorporated herein by reference in its entirety. However, other methods of obtaining phenolic acid extracts from berries are possible and encompassed within the scope of the present disclosure.

As described in the examples herein, it has been found that extracts of berries from the Vaccinium family are useful for enhancing or promoting wound healing, and such extracts may be formulated in a gel formulation, in a cream formulation, or in a nanocellulose hydrogel formulation. As shown in the examples herein, these formulations are advantageous for wound healing and other processes such as collagen remodeling. Thus, in accordance with the present disclosure, provided herein are compositions and methods involving extracts from berries of the Vaccinium family that are useful for promoting, improving, enhancing, or stimulating wound healing. However, it is understood that in any of the formulations or compositions described herein, the extract may alternatively be one or more phenolic acids instead of phenolic acid compounds which were literally extracted from a berry. In other words, provided herein are compositions comprising one or more phenolic acids and pharmaceutically acceptable adjuvants, diluents, and/or carriers, and the use of such compositions for promoting wound healing or other processes such as collagen remodeling.

In a first aspect, provided herein is a composition in the form of a cream that includes phenolic acids. The phenolic acids may be from an extract of a berry from the Vaccinium family, but do not need to be from a berry extract. In one non-limiting example, the cream composition includes the phenolic acid extract, water, shea butter glycerides, phenethyl alcohol, castor seed oils, cetearyl alcohol, glyceryl stearate, sodium stearoyl lactylate, and balsam peru oil.

In another aspect, provided herein is a composition in the form of a serum or gel that includes phenolic acids. The phenolic acids may be from an extract of a berry from the Vaccinium family, but do not need to be from a berry extract. In one non-limiting example, the serum or gel composition includes the phenolic acid extract, water, phenethyl alcohol, hydrolyzed hyaluronic acid (HA), and sodium hyaluronate. As shown in the examples herein, the serum/gel composition is capable of promoting wound closure by increasing vascularization, re-epithelialization, and collagen production, and is anti-inflammatory. The natural ingredients containing wild blueberry extracts have the properties to promote faster wound healing by increasing tissue-regeneration and repair and reducing inflammation. The serum/gel composition is an all-natural product with a toxic-free formulation, free from synthetic chemicals, environmentally friendly, and low-cost.

In another aspect, provided herein is a composition in the form of a hydrogel that includes phenolic acids and nanocellulose. The hydrogel may be a nanocellulose hydrogel such as, but not limited to, a TEMPO-oxidized CNF hydrogel. The TEMPO-oxidized CNF hydrogel may be crosslinked with a suitable crosslinker such as citric acid. However, other nanocellulose hydrogels are possible and encompassed within the scope of the present disclosure. The phenolic acids may be from an extract of a berry from the Vaccinium family, but do not need to be from a berry extract. In one non-limiting example, the hydrogel composition is a citric acid crosslinked TEMPO-oxidized CNF hydrogel with a phenolic acid extract containing chlorogenic acid, caffeic acid, and ferulic acid. In other non-limiting examples, the hydrogel composition is a salt-crosslinked TEMPO-oxidized CNF hydrogel with a phenolic acid extract containing chlorogenic acid, caffeic acid, and ferulic acid. The salt may be, for example, NaCl or CaCl₂).

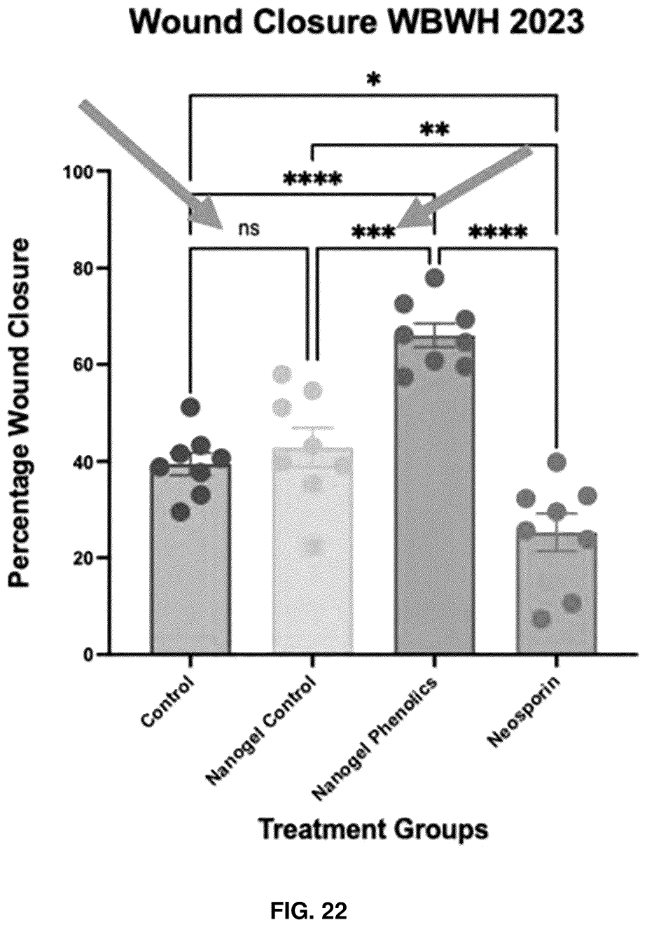

As shown in the examples herein, a TEMPO-oxidized CNF hydrogel composition with the phenolic acid extract from wild blueberries is capable of promoting wound healing better than a negative control (nothing on the wound), better than the CNF hydrogel without the phenolic acid extract, and better than a positive control (Neosporin) (FIG. 22). The composition can promote wound healing to a higher degree, and is superior to, the most commonly used drug on the market, Neosporin. This is also an all-natural product with a toxic-free formulation, free from any synthetic chemicals, and which is environmentally friendly and low-cost. The natural ingredients containing wild blueberry extract have the properties to promote faster wound healing by increasing tissue regeneration and repair and reducing inflammation.

Hydrogels are three-dimensional networks of hydrophilic polymers that can absorb and retain a significant amount of water. Cellulose nanofibril (CNF) hydrogels are materials derived from cellulose, a natural biopolymer found in the cell walls of plants. CNF can be used to form hydrogels through various methods such as, but not limited to, physical cross-linking, chemical cross-linking, or a combination of both. CNF hydrogels have a closer fiber aggregation compared to CNF due to the addition of salts.

CNFs are nanoscale fibers that can be extracted from various cellulose-rich sources, such as wood, cotton, or other plant materials. CNFs have a high aspect ratio, have a high surface area, and are biodegradable. CNFs can be obtained through mechanical or chemical treatments applied to cellulose-rich sources. Mechanical methods may involve grinding or homogenizing the cellulose material to break it down into nanoscale fibrils, while chemical methods use specific treatments to isolate the nanofibrils. Chemical treatments used to obtain CNF may include acid hydrolysis, oxidative treatments, enzymatic hydrolysis, or alkaline treatments. As one non-limiting example, an oxidative treatment may involve the use of oxidizing agents, such as sodium hypochlorite (NaClO) or 2,2,6,6-tetramethylpiperidine-1-oxyl (TEMPO), optionally in combination with a mechanical treatment. In such an oxidative treatment, the oxidizing agents modify the surface of the cellulose fibers, making them more susceptible to mechanical disintegration into nanofibrils. Oxidative treatments can yield CNFs with improved surface functionalities.

When CNF is obtained by a TEMPO process, the CNF has distinctive characteristics compared to CNFs obtained through other methods. The TEMPO process involves the use of a radical initiator (TEMPO) and NaClO to selectively oxidize the primary hydroxyl groups of cellulose. This results in CNF having surface functionalization (due to the introduction of carboxylate groups on the CNF surface, which imparts negative charges to the CNFs, preventing their aggregation), colloidal stability (from the carboxylate groups, which cause electrostatic repulsion), biocompatibility, and improved purity. In accordance with the present disclosure, TEMPO-oxidized CNF is particularly useful for the creation of CNF hydrogels to be used as drug carriers.

TEMPO oxidized CNF hydrogels have been used in various veterinary and biomedical applications due to their bioinert nature. Salt bridging enhances the mechanical properties of TEMPO including stability in aqueous solutions. Salts shield the negative charge of TEMPO fibers allowing for closer aggregation of fibers. Closer fiber aggregation and salt bridging results in improved gel strength. However, it is understood that CNFs obtained from non-TEMPO process may also, or alternatively, be used to form the CNF hydrogels described herein, and used as drug carriers in formulations with one or more phenolic acids.

CNF hydrogels can absorb and retain large amounts of water. This is advantageous for drug delivery as it can help in solubilizing and delivering hydrophilic drugs effectively. CNF hydrogels may be transparent, making them suitable for certain optical or medical applications. CNF hydrogels can be used for drug delivery because of their biocompatibility and ability to mimic the extracellular matrix. CNF hydrogels are biocompatible and can be well-tolerated by the body, which is important for adjuvant applications as it ensures that the adjuvant does not induce adverse reactions or toxicity.

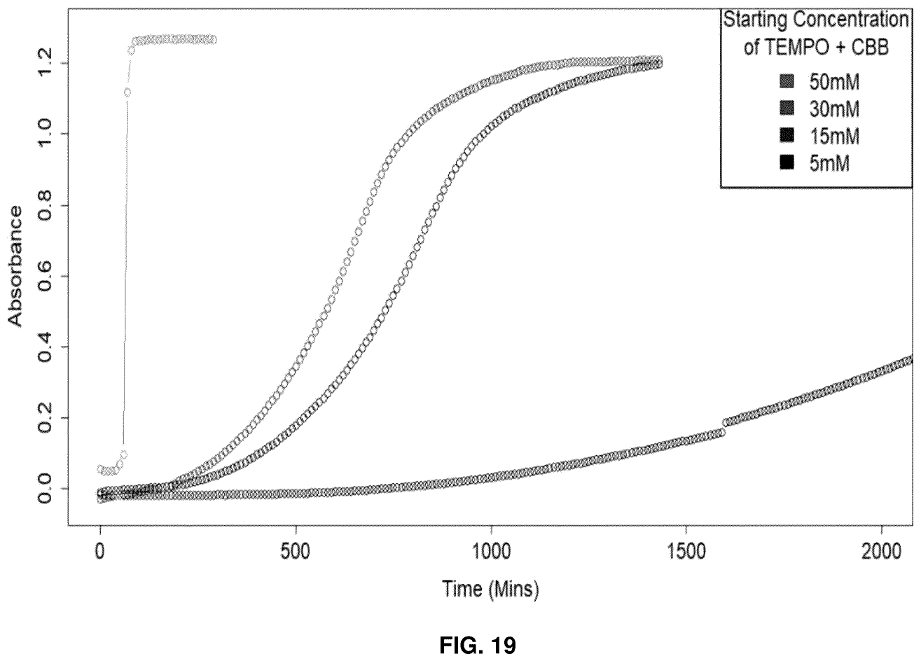

CNF hydrogels can provide sustained release of drugs over time. The three-dimensional network structure of the hydrogel formed by the CNF allows for controlled diffusion of drugs from the gel matrix. The examples herein demonstrate the controlled diffusion of a dye from a CNF hydrogel (FIG. 19). CNF hydrogels can encapsulate various types of drugs, including both hydrophilic and hydrophobic compounds. This versatility makes CNF hydrogels suitable for a wide range of pharmaceutical applications.

It may be difficult to crosslink pure CNF to form a hydrogel. However, modified CNF, such as TEMPO-oxidized CNF, is easier to form a hydrogel. Suitable crosslinkers for forming a hydrogel with TEMPO-oxidized CNF include, but are not limited to, polyethyleneimine, metals (such as Fe³⁺, Ca²⁺, or Na⁺), citric acid, dialdehydes, acetals, polycarboxylic acids, and epichlorohydrin/polyepichlorohydrin. A TEMPO-oxidized CNF hydrogel may be prepared by mixing a crosslinker such as citric acid or a salt with TEMPO-oxidized CNF to crosslink the TEMPO-oxidized CNF. The salt may be, for example, NaCl or CaCl₂). However, other crosslinkers are possible and encompassed within the scope of the present disclosure.

Furthermore, carboxylic acid groups present on TEMPO-oxidized CNF are easily conjugated through an amide bond. This amide bond occurs when an amine group (—NH₂) reacts with a carboxylic acid group (—CONH), causing the amine group to bond to the carboxylic acid. This process of amide bonding onto the carboxylic acid is referred to as amidation. Through this process, the overall characteristics of the TEMPO-oxidized CNF are altered without compromising the overall solubility of the polymer. The amidation of TEMPO-oxidized CNF acts as a reinforcing structure and improves the dispersibility of cellulose. The amidation of TEMPO-oxidized CNF may create a branched polymer system and an injectable shear thinning gel. Once the gel is injected and shear forces are removed from the system, the aligned amidated side chains may entangle to recreate the matrix in the peritoneal cavity, allowing for long-term diffusion. The process of amidating TEMPO-oxidized CNF may utilize toxic residual chemicals, but sufficient washing of the hydrogel may remove them. In some embodiments, the CNF-based hydrogels described herein include amidated TEMPO-oxidized CNF.

Any of the cream, gel/serum, or hydrogel compositions described herein may optionally include one or more additional adjuvants, diluents, carriers, fragrances, or other additives. Such other additives may include, but are not limited to, one or more antibiotics, anti-inflammatory agents, antiseptics, growth factors, hyaluronic acid, collagen, enzymes, vitamins or minerals, pain relievers, emollients, surfactants, or preservatives.

A non-limiting example method for making a nanocellulose hydrogel with the blueberry extract may include extracting phenolics from berries, measuring the concentration of the phenolic acid extract and diluting it to 1000 μg/ml, obtaining TEMPO-oxidized CNF, adding 500 μg/ml phenolic acid extract to 5 mls of 1000 μg/ml phenolic acid in pierce water (LC-MS grade tincture) and 9 grams of TEMPO-oxidized CNF along with 1 gram of citric acid, mixing together on a stirrer until properly mixed with a consistent appearance (about 1-2 minutes), adding a desired volume of the mixture to a container to form the desired shape, and placing the container in 4° C. for 24 hours and covering to maintain moisture until use. To form the desired shape, the mixture can be left uncovered for 48 hours at 4° C. This produces a nanogel with 500 μg/ml phenolic acid extract.

A non-limiting example TEMPO-oxidized CNF hydrogel composition can be made by weighing out a desired amount of 1.1% TEMPO-oxidize CNF (TCNF) solution and vacuum filtering the TCNF solution until it reaches a weight percentage of 3.6%. Then, the phenolic acid extract can be added to a quantity of the TCNF solution and mixed until thoroughly combined, followed by the addition of a desired amount of a crosslinker solution and mixing to form a gel. The concentration of crosslinker used may vary. In one non-limiting example, a 1500 μM citric acid solution is used to prepare the hydrogel. The amount of CNF present in the hydrogel may range from about 0.5 wt % to about 5.0 wt %. In non-limiting examples, the CNF is present in the hydrogel in an amount of about 1.1 wt % or an amount of about 3.6 wt %. The concentration of the phenolic acid extract may range from about 200 μg/ml to about 1500 μg/ml. In non-limiting examples, the phenolic acid extract is present at a concentration of about 500 μg/ml or a concentration of about 1000 μg/ml. However, other amounts and relative amounts of the components of the composition are possible and encompassed within the scope of the present disclosure.

The compositions described herein can be used for treating various wounds such as, but not limited to, diabetic foot ulcers, pressure ulcers, venous leg ulcers, burns, severe trauma, post-surgical wounds, and scarring. The compositions are also useful to treat minor scrapes, cuts, and burns (in children and adults, including athletes), iritis, oral wounds, and periodontal disease. Such wounds can be treated by delivering the compositions topically or transdermally. Additionally, the compositions may be useful in cosmetic formulations for skin regeneration and repair. In contrast to the compositions described herein, conventional products on the market for wound care are typically anti-microbials (such as Neosporin) which primarily prevent infection but do not speed wound closure, revascularization, re-epithelialization, or collagen formation. The compositions are superior to conventional alternatives because they deliver bio-derived (naturally sourced) bioactive compounds that speed up wound closure, reduce inflammation, and minimize patient discomfort. The most widely used drug on the market for wound healing, Neosporin, is shown in the examples herein to be inferior in speeding up wound closure.

Pharmaceutical compositions of the present disclosure may include an effective amount of a phenolic acid extract from a berry of the Vaccinium family, a cream, gel, or hydrogel, and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered, for example, topically, or by other methods for promoting wound healing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) (i.e., a phenolic acid extract) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. It may be stable under the conditions of manufacture and storage and may be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption such as, for example, aluminum monostearate or gelatin.

Pharmaceutical compositions for topical administration may include the compositions formulated for a medicated application such as an ointment, paste, cream, gel, serum, hydrogel, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

The compositions disclosed herein may also be delivered from a bandage or other substrate configured to be adhered to skin. The compositions can be encapsulated or dispersed within a matrix material of the bandage, allowing for sustained and localized delivery. Various polymers, adhesives, and permeation enhancers may be included in the bandage material to facilitate permeation of the compositions through the skin. The bandage can be designed to adhere securely to the skin, providing a convenient and non-invasive mode of administration.

The compositions disclosed herein may also be incorporated into various cosmetic or nutraceutical products, such as for skin rejuvenation or for the treatment of, for example, periodontal disease, oral wounds, iritis, minor cuts or abrasions, surgical wounds, burns, pressure ulcers, diabetic foot ulcers, postsurgical scar healing, chronic wounds, or radiation-induced skin injuries. Nutraceutical products may further include one or more vitamins (such as vitamins A, C, or E), minerals, amino acids, herbal extracts, protein, omega-3 fatty acids, or other natural compounds in addition to the phenolic acid extract. A cosmetic product including the phenolic acid extract may be used, for example, for scar reduction, wound soothing, moisturization, or antioxidant protection. Such cosmetic products may further include one or more preservatives, emollients, surfactants, thickeners, humectants, colorants, fragrances, antioxidants, or UV filters.

It is further envisioned that the compositions and methods described herein can be embodied in the form of a kit or kits. A non-limiting example of such a kit is a kit for preparing a composition for promoting wound healing, the kit comprising a TEMPO-oxidized CNF hydrogel and a phenolic acid extract from a berry of the Vaccinium family in separate containers, where the containers may or may not be present in a combined configuration. Many other kits are possible. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

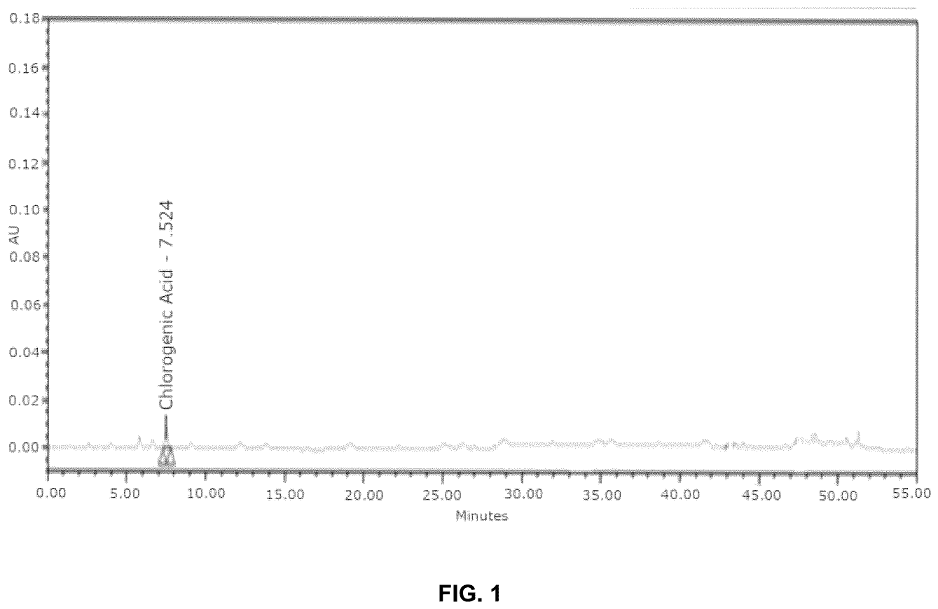

The examples herein demonstrate that a phenolic extract derived from wild blueberries has the ability to significantly promote wound healing. For all the examples herein, the blueberry extract referred to as the “phenolic extract”, or “PA”, contained mainly chlorogenic acid (10.23±1.8 mg/mL) with traces of ferulic and caffeic acids (FIG. 1). FIG. 1 shows an HPLC profile analysis of the PA fraction of wild blueberry (V. angustifolium) powder.

### Example I—Phenolic Extracts from Wild Blueberries Promote Collagen Remodeling During Wound Healing

Collagen remodeling is important for adequate wound healing. The amount, thickness, and arrangement of collagen within wounds determines the strength of the skin. Effective wound remodeling is a significant challenge associated with chronic wounds such as diabetic and pressure ulcers. Polyphenols from wild blueberry can improve wound healing via endothelial cell migration in vitro. In this example, the role of phenolics and a mixture of phenolics and anthocyanins extracted from wild blueberries on collagen remodeling in vivo is examined.

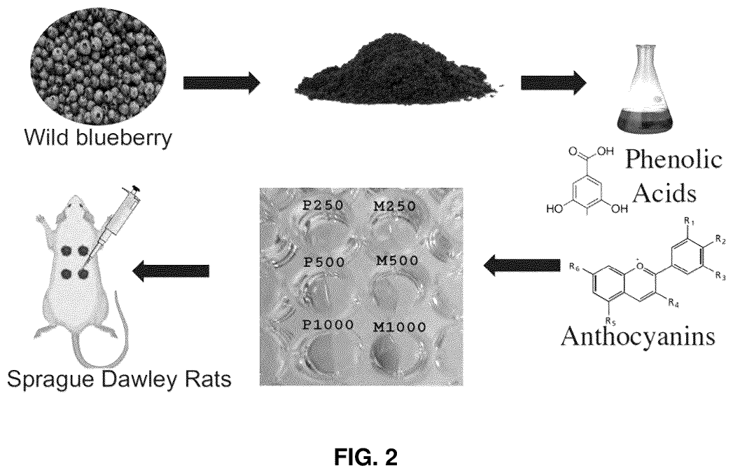

The experimental design is shown in FIG. 2. Phenolics (P) and mixed (M) (i.e., phenolic and anthocyanin) fractions were extracted from wild blueberries by solid phase extraction. The extracts were resuspended in distilled water based on treatment groups and applied to four dorsal locations of 6 mm wound punches on rats.

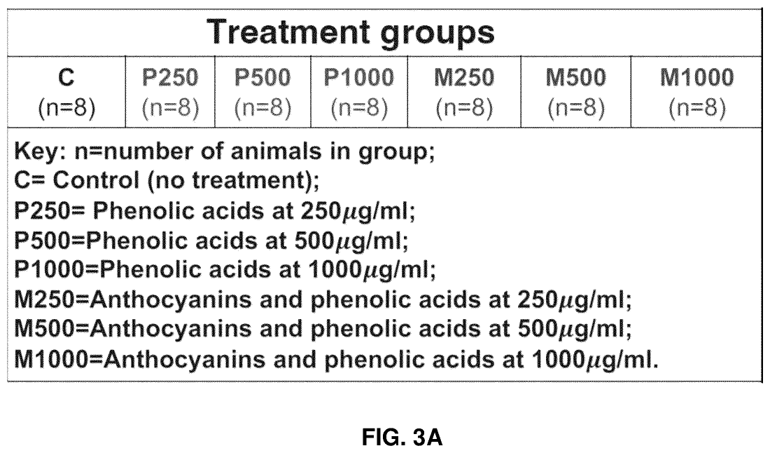

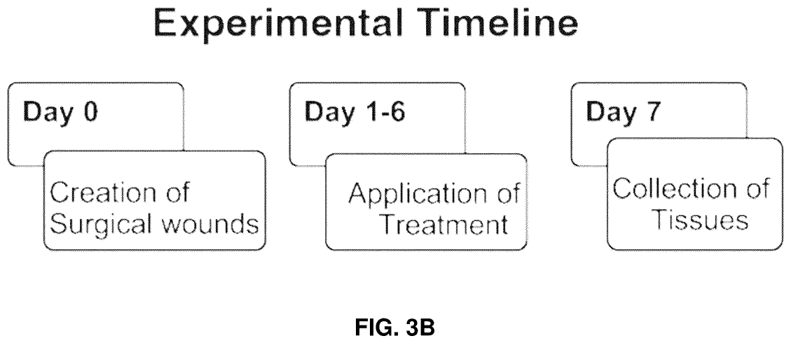

FIG. 3A shows the treatment groups studied and FIG. 3B shows the experimental timeline. Dorsal wounds were created on the rats and treated for 7 days according to the treatment groups. Wound tissues were excised for histological analysis. Surgical wounds were created at day 0, the treatment was applied during days 1-6, and tissues were collected on day 7.

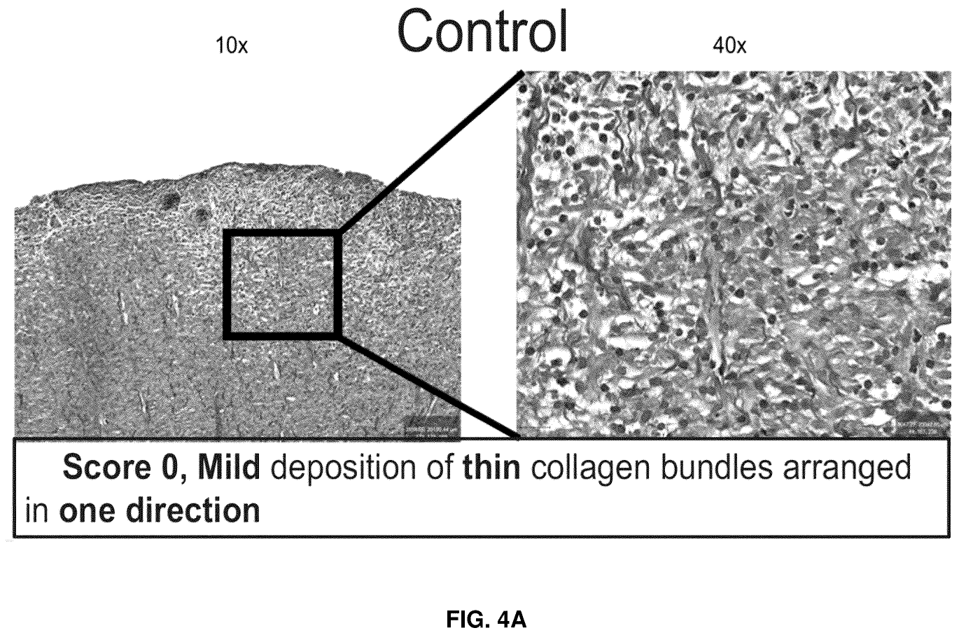

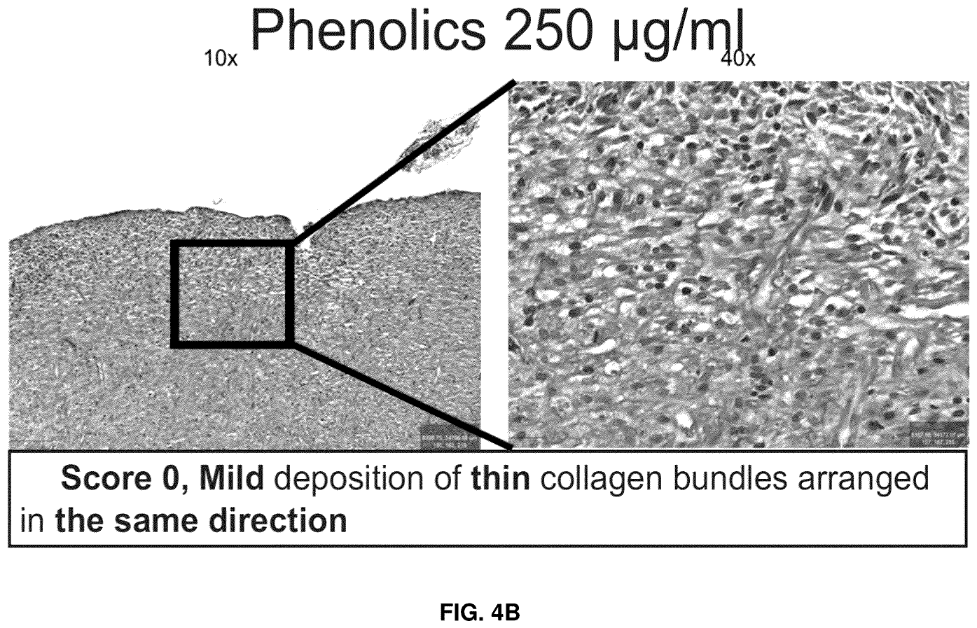

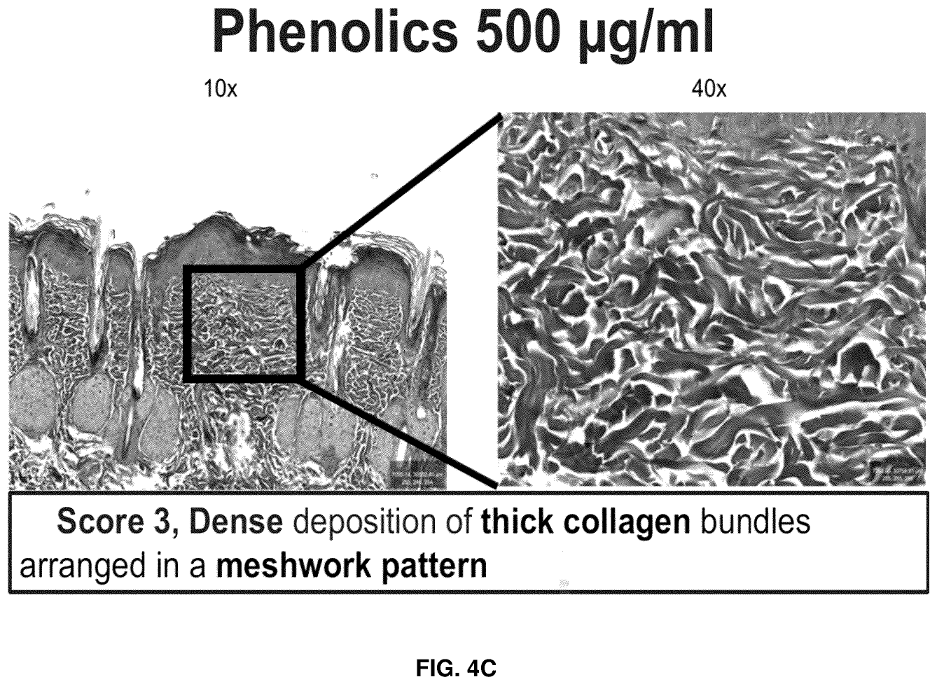

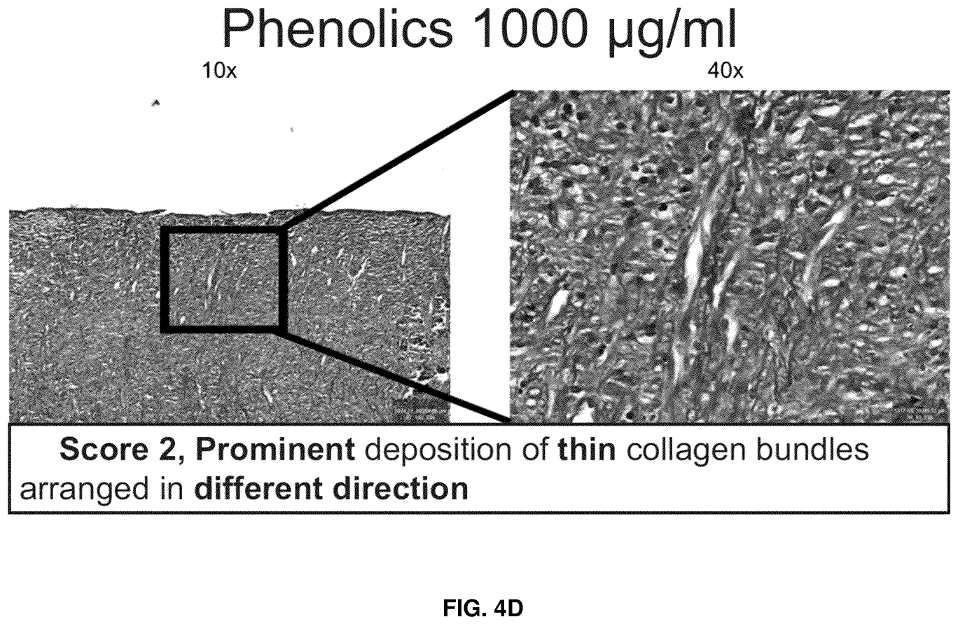

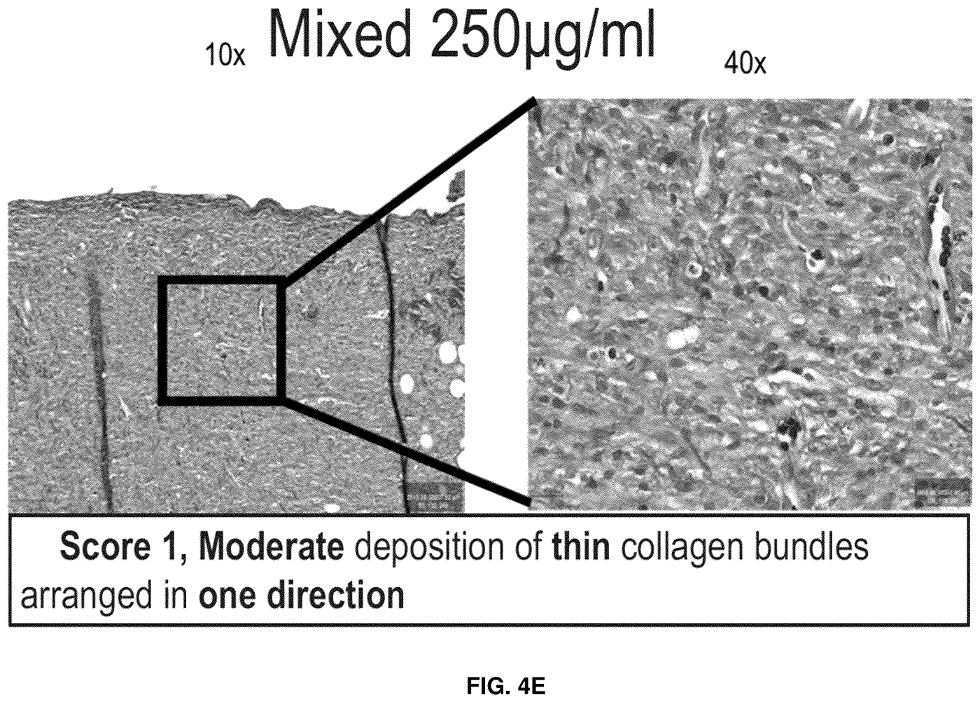

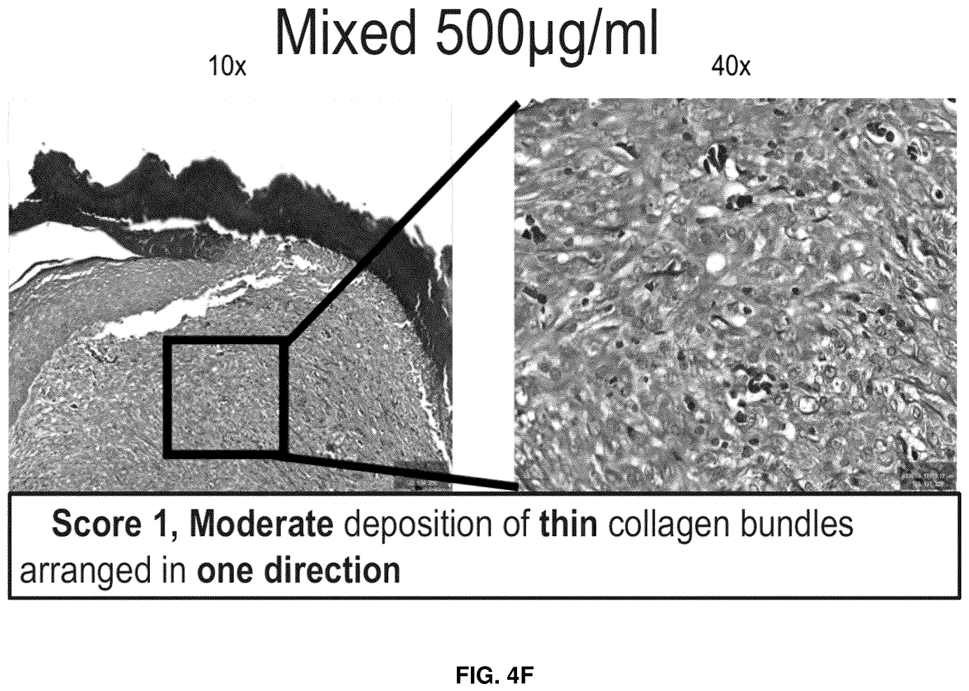

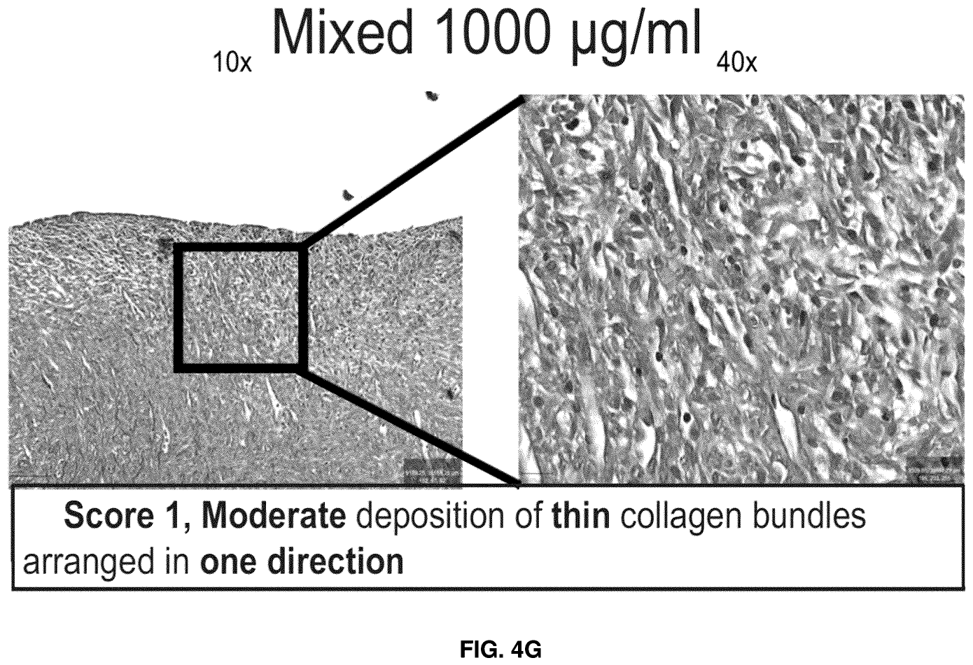

Tissues were fixed, stained, imaged, and observed for collagen quality using Qu path software at 10× and 40×. FIGS. 4A-4G show the results. The following Table 1 displays the collagen remodeling scores used.

TABLE 1
Collagen remodeling score

| Score | Parameters |
|---|---|
| 0 | Mild deposition of collagen |
| 1 | Moderate deposition of collagen |
| 2 | Prominent deposition of collagen |
| 3 | Dense deposition of collagen |

The collagen fibers in the P500 μg/ml treatment group were thick, dense, and arranged in a meshwork pattern while the other treatments resulted in thin fibers arranged in one direction, perpendicular to the epidermis. This result agrees with the ability of phenolics at 0.002 and 60 μg/ml to significantly promote increased angiogenesis and wound closure. Increased speed of wound closure and enhanced vascularization have also been demonstrated with P500 μg/ml treatment.

The results of this example show that complete remodeling occurred in the P500 μg/ml group with thick and dense collagen fibers arranged in a meshwork pattern when compared with the control. Thus, phenolic acid extracts from wild blueberries are useful for promoting wound healing, such as in patients suffering from chronic wounds (e.g., diabetic wounds).

### Example II—Phenolic Acids from Wild Blueberries in Serum/Gel Facilitate Wound Healing Via Vascular Remodeling

Complete wound re-epithelialization (WRE) is important for wound healing. However, epidermal re-epithelialization is a major challenge in chronic wounds such as diabetic and ischemic ulcers. As previously demonstrated, significant angiogenesis is observed in HUVEC cells when treated with a phenolic acid extract from wild blueberries. Furthermore, significant wound closure has been observed with phenolic fractions from wild blueberry in a rat in vivo model. This example describes an evaluation of the vascular remodeling effects of a phenolic extract from wild blueberry on wound healing pathways in a rat model. Specifically, this example describes the effects of the phenolic extracts at 500 μg/ml and 1000 μg/ml in a serum (gel) and a cream on percentage wound closure, the histology of vascularization and collagen deposition, and the proteomic expression of vascularization and collagen deposition.

### Materials and Methods

The serum and gel compositions were prepared once a week. The formulas provided enough treatment for six days for one animal with four 6 mm dorsal full-thickness wounds.

500 μg/ml phenolic acid in pierce water were added to LC-MS grade tincture Pierce Water, 0.2% wound healing grade HA (100-300 kDa molecular weight), 1.5% gelling grade HA (800-1,200 kDa molecular weight), and 1.0% phenethyl alcohol (as a preservative). This produced the serum base for the P500 μg/ml serum/gel having a pH of 5.5.

1000 μg/ml phenolic acid in pierce water were added to LC-MS grade pierce water, 0.2% wound healing grade HA (100-300 kDa molecular weight), 1.5% gelling grade HA (800-1,200 kDa molecular weight), and 1.0% phenethyl alcohol (as a preservative). This produced the serum base for the P1000 μg/ml serum/gel having a pH of 5.5.

To obtain the P500 μg/ml serum/gel, 250 μl of 2000 μg/ml phenolic acid in pierce water, LC-MS grade tincture were added to 750 μg of the serum base. The tincture and serum base mixture was stirred in an amber glass 2 mL jar, mixed at 300-500 RPM for 15 minutes to ensure a uniform mixture, and kept at 4° C. until use.

To obtain the P1000 μg/ml serum/gel, 500 μl of 2000 μg/ml phenolic acid in pierce water, LC-MS grade tincture were added to 500 μg of the serum base. The tincture and serum base mixture was stirred in an amber glass 2 mL jar, mixed at 300-500 RPM for 15 minutes to ensure a uniform mixture, and kept at 4° C. until use.

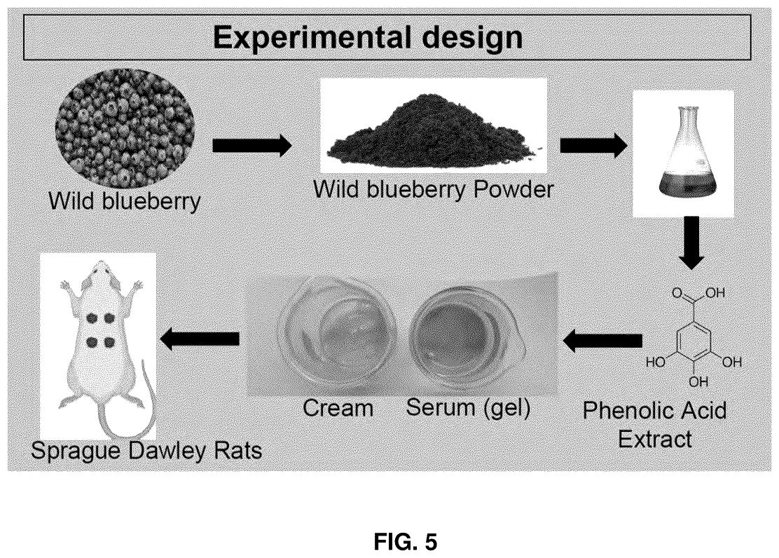

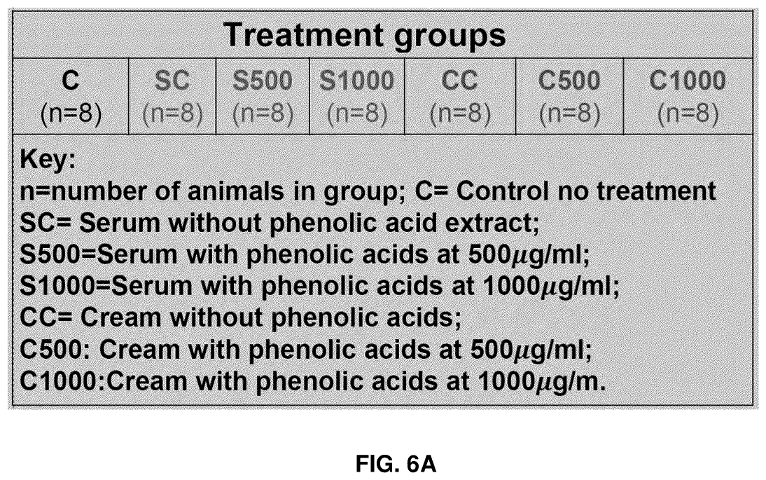

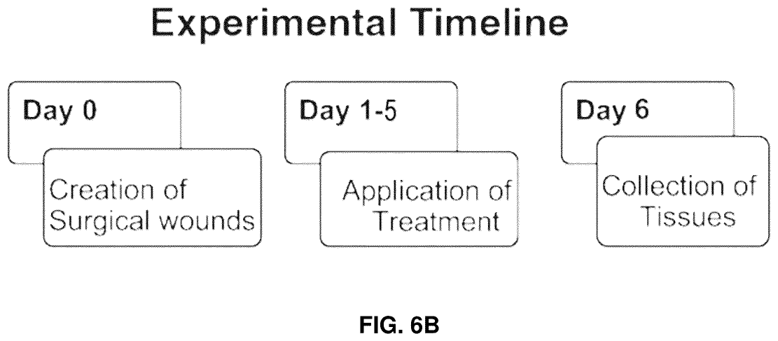

FIG. 5 illustrates the experimental design used. FIG. 6A shows the treatment groups studied and FIG. 6B shows the experimental timeline evaluated. Tissues were stained with H&E and MT, and imaged with the MoticEasyScan Infinity 60 microscope. Images were analyzed using Qu-path 0.2.3 at 10×, 20×, and 40× objectives. For the histology analysis of re-epithelialization, images were analyzed using Qu-path 0.2.3 at 5× objectives. Endothelial cells were counted and analyzed as one-way ANOVA in prism while collagen deposition was scored as qualitative organization according to the conventional method.

Percentage wound closure is calculated according to the following equation: Percentage wound closure (%)=[1−(final wound area/initial wound area)]×100

Photographic data were used to calculate the percentage wound closure using ImageJ software. Data were analyzed as one-way ANOVA in prism. The same collagen remodeling scores defined in Table 1 in Example I above were used. In addition, vascularization scores were assigned according to the following definitions in Table 2.

TABLE 2
Vascularization score

| | |
|---|---|
| Mild Vascularization | Presence of few endothelial cells |
| Moderate Vascularization | Presence of moderate endothelial cells |
| High Vascularization | Presence of abundantly defined endothelial |
| | cells |

The epidermal layer of the skin was examined for regeneration and scored as wound re-epithelialization according to the scores defined in the following Table 3.

TABLE 3
Re-epithelialization score for epidermal regeneration

| Score | Parameters |
|---|---|
| 0 | Mild/none epithelializaton |
| 1 | Discrete/little eptihelialization with a small epithelial layer (the |
| | epithelial tongue occupies the edge of the wound, at most, ⅓ of |
| | the wound gap) |
| 2 | Moderate/partial epithelialization with a longer new epithelial layer |
| | (the epithelial tongue occupies the edge of the wound more than ⅓ |
| | of the wound gap) |
| 3 | Complete/whole epithelialization |

For the proteomic analysis of re-epithelialization, tissues were lysed, run in SDS-PAGE gel, and transferred onto blots according to Bio-Rad protocol. Blots were incubated with primary antibodies: epithelial growth factor (EGFR) and fibroblasts growth factor (FGF7) with their corresponding secondary antibodies. Blots were imaged with a LICOR Odyssey CLx machine for qualitative protein expression. Densitometry was measured with image J software and analyzed with one-way ANOVA in prism (prism v9).

Results from Blueberry Extracts in Serum/Gel or Cream

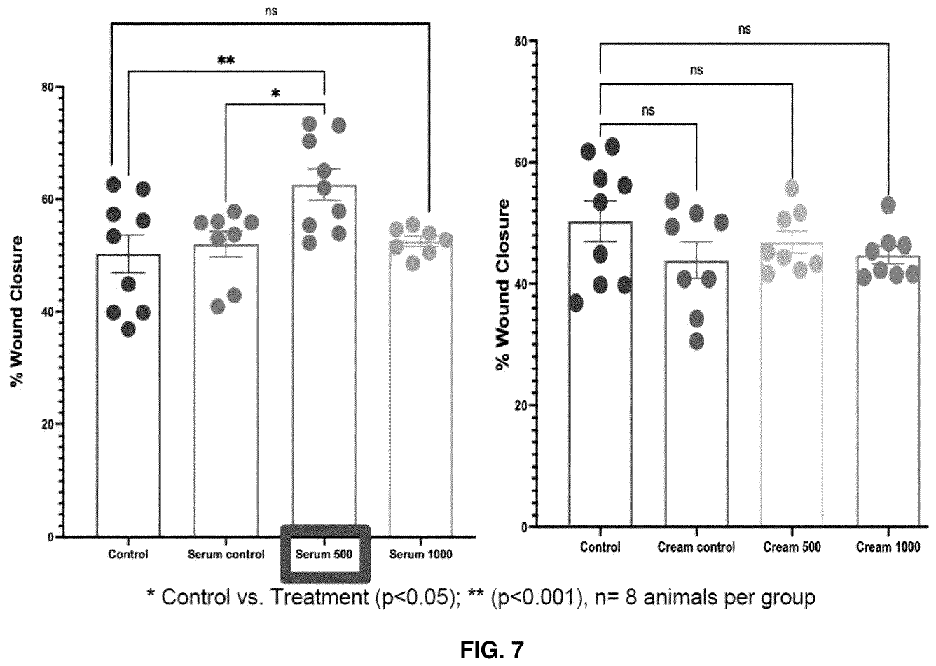

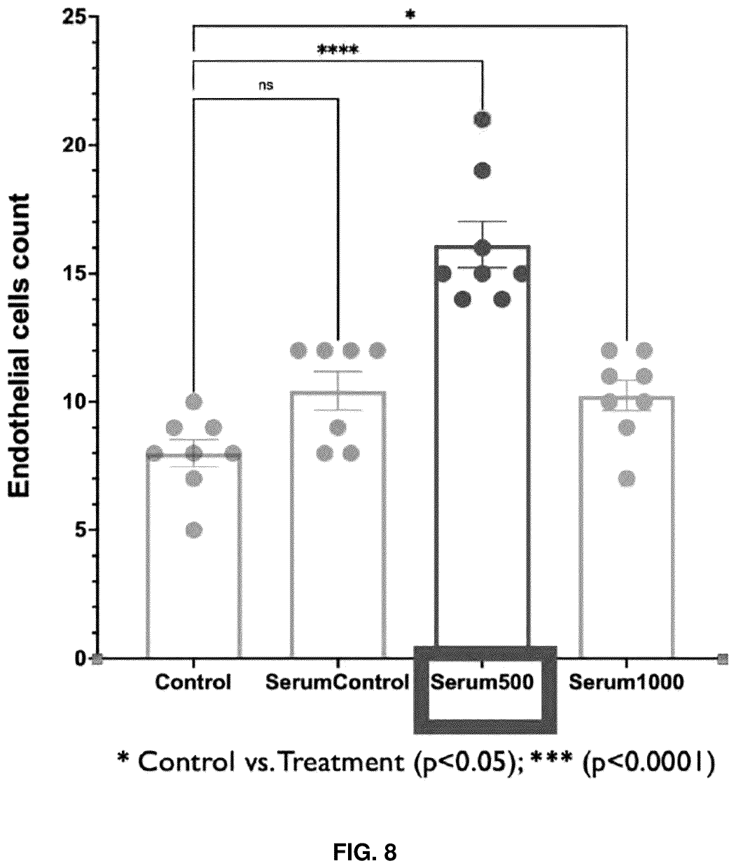

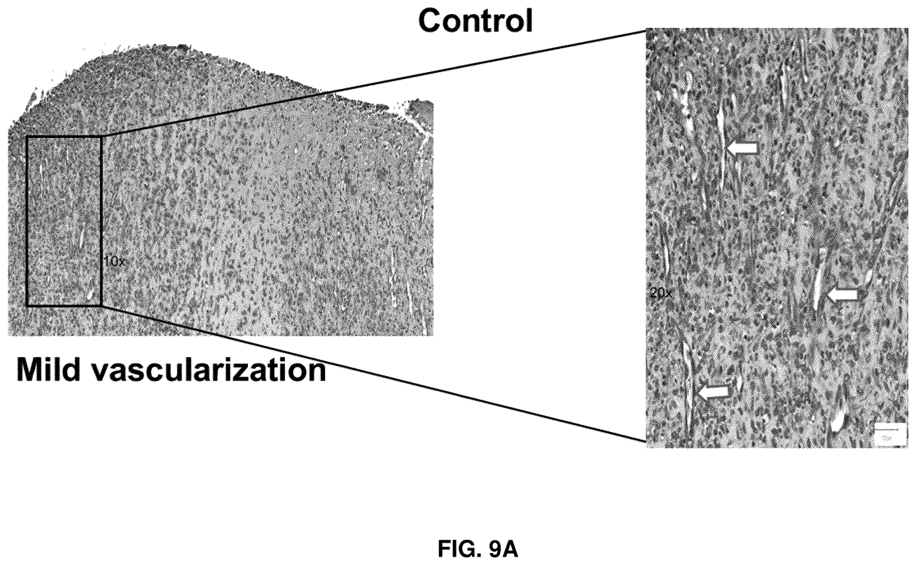

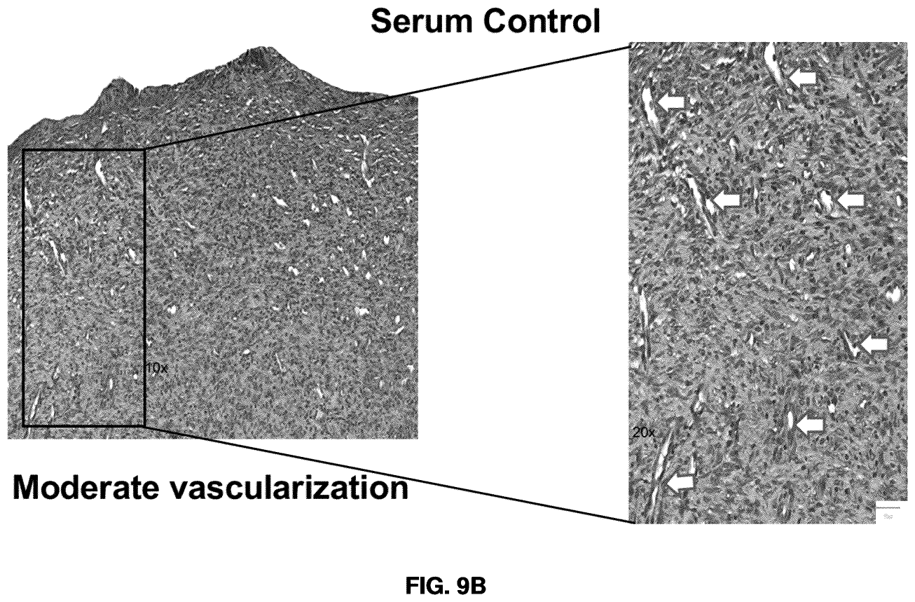

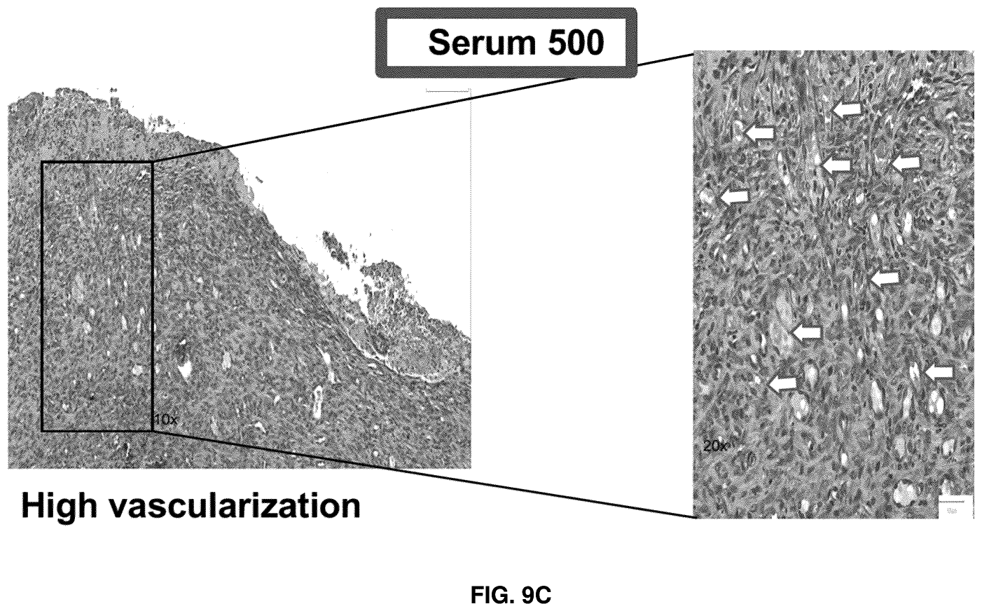

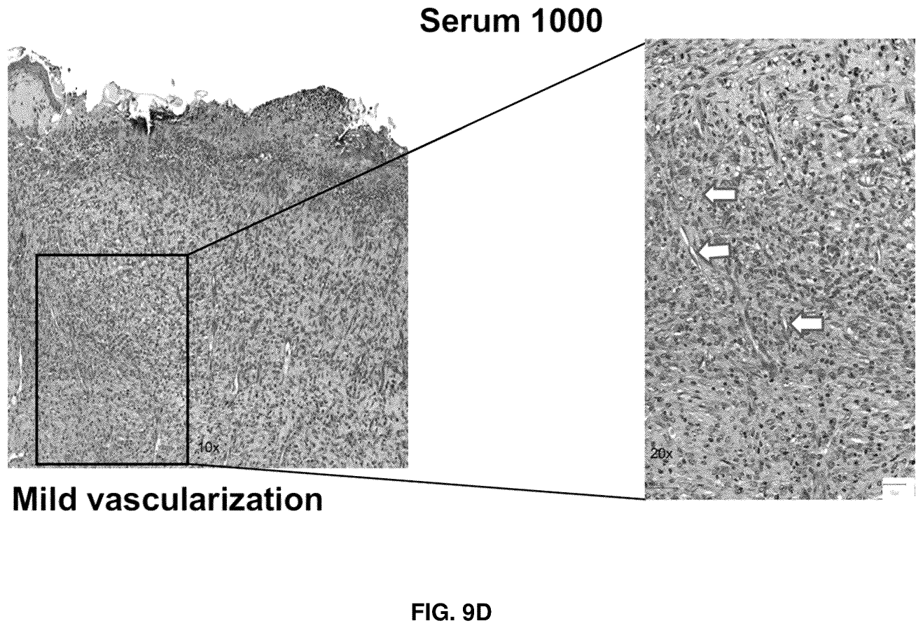

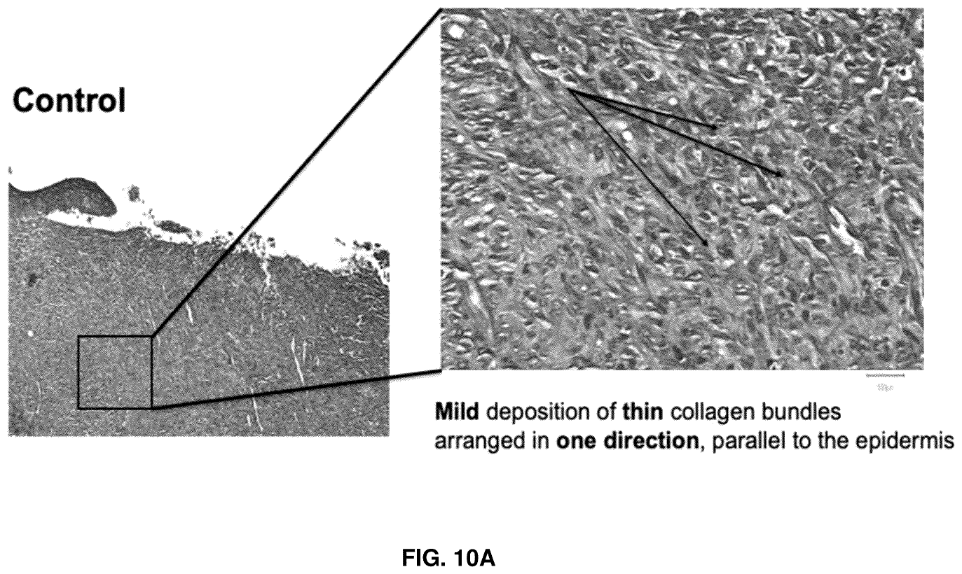

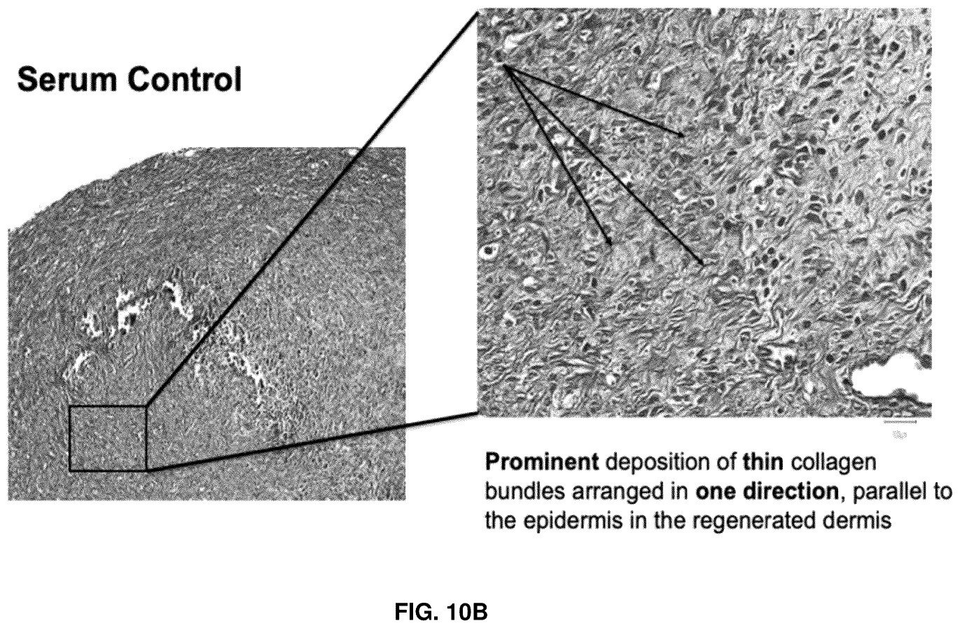

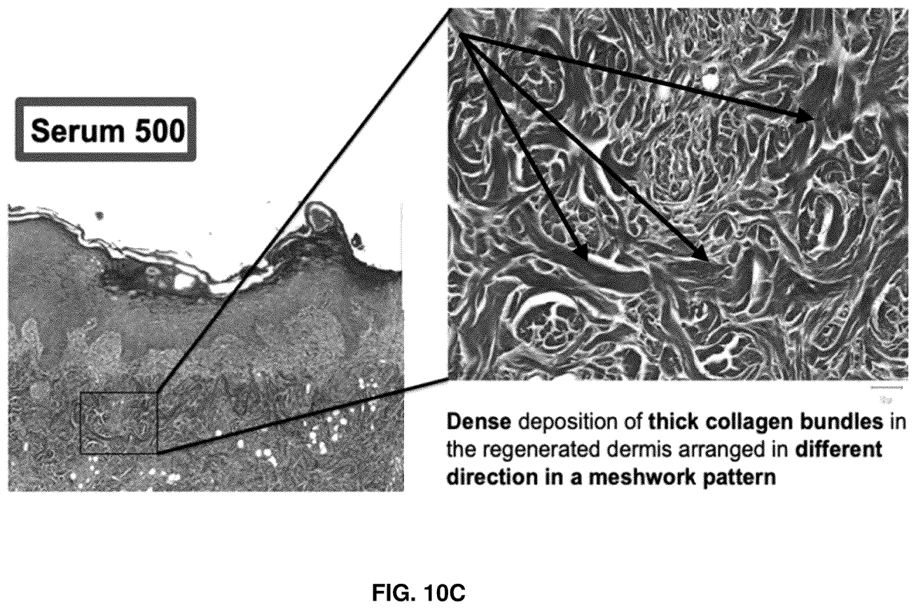

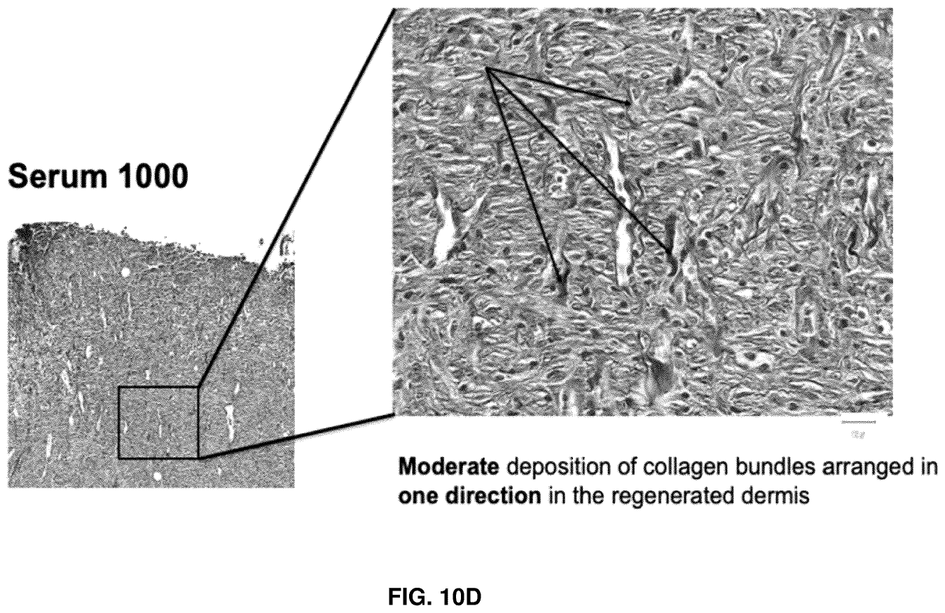

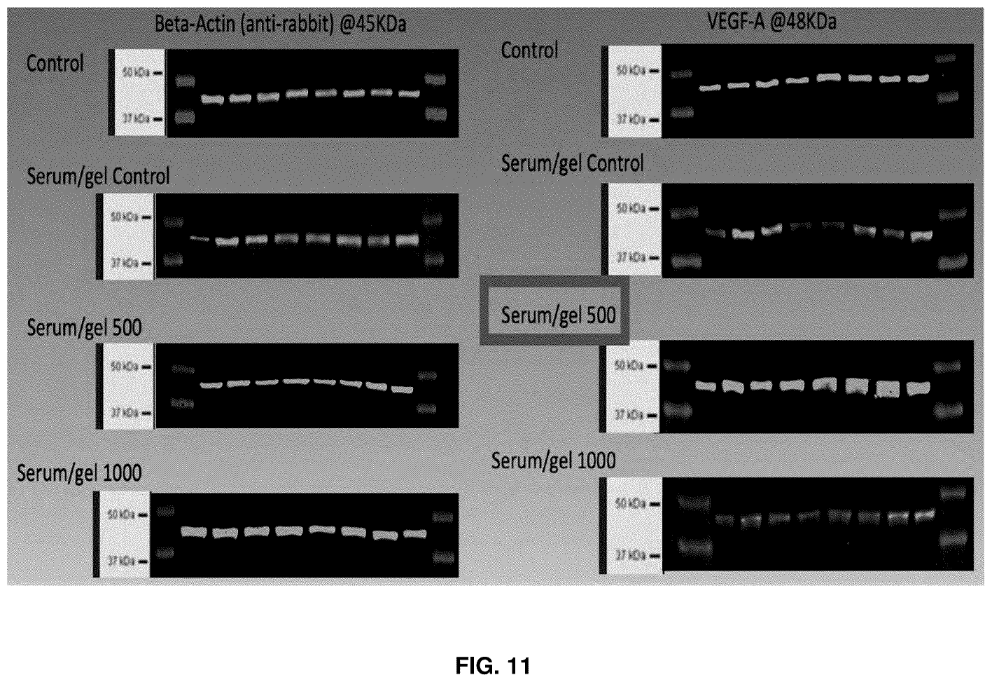

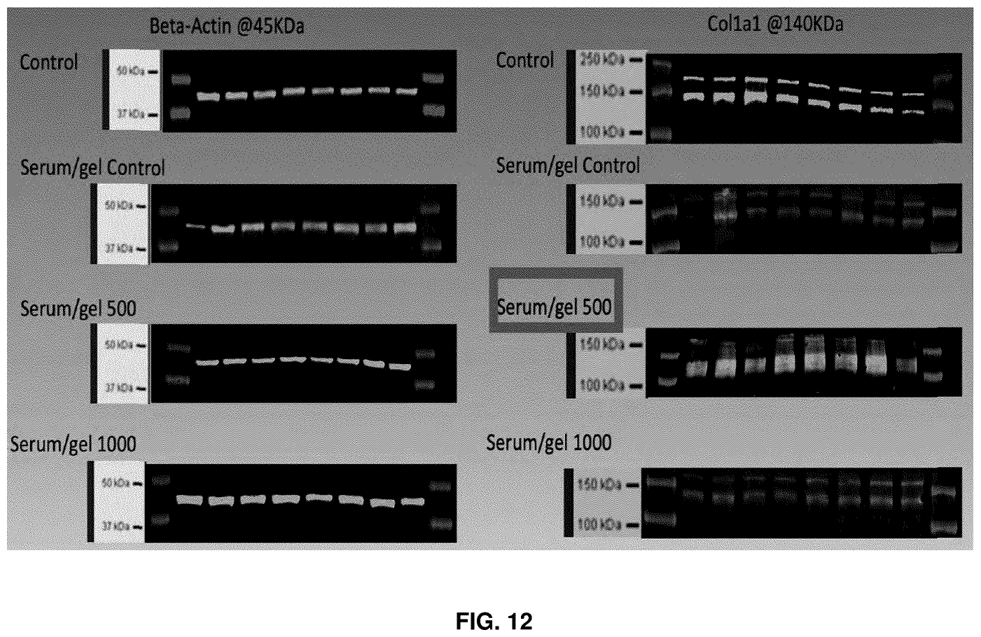

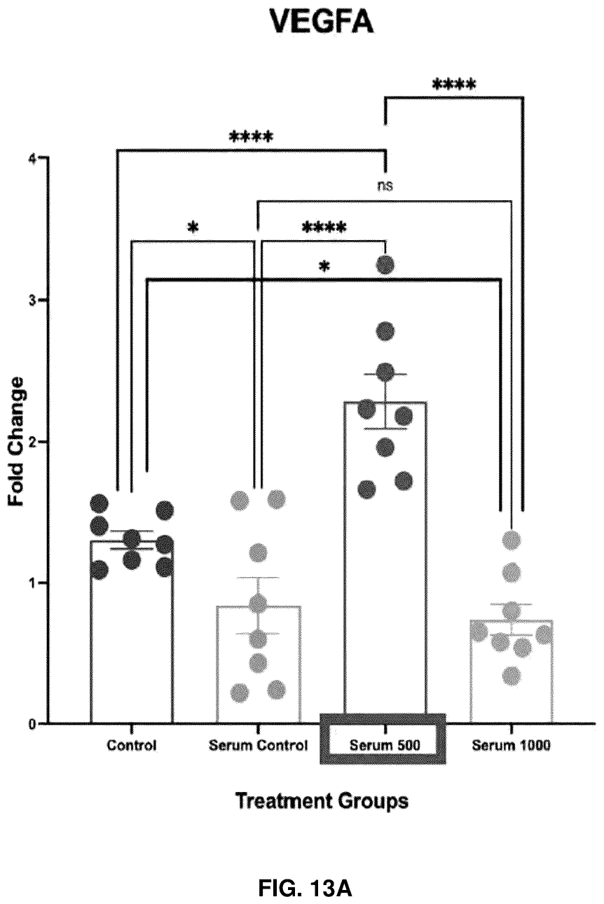

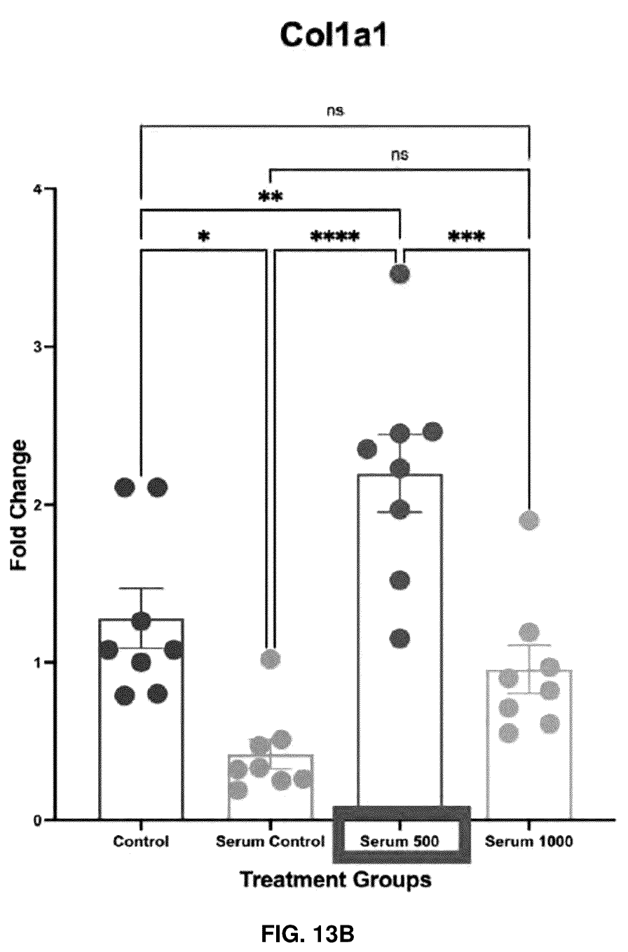

FIG. 7 shows the results of wound closure from the serum/gel (500 and 1000 μg/ml) and from the cream (500 and 1000 μg/ml) versus a control, and FIG. 8 shows the endothelial cells count for the serum/gel (500 and 1000 μg/ml) versus a control. The vascularization score results are shown in FIGS. 9A-9D. The collagen remodeling score results from the serum/gel are shown in FIGS. 10A-10D. The vascular protein expression of vascular endothelial growth factor A (VEGFA) from the serum/gel is shown in FIG. 11. The collagen protein expression of collagen 1a1 (Col1a1) from the serum/gel is shown in FIG. 12. FIGS. 13A-13B show the fold change in expression of VEGFA and Col1a1 relative to beta-actin.

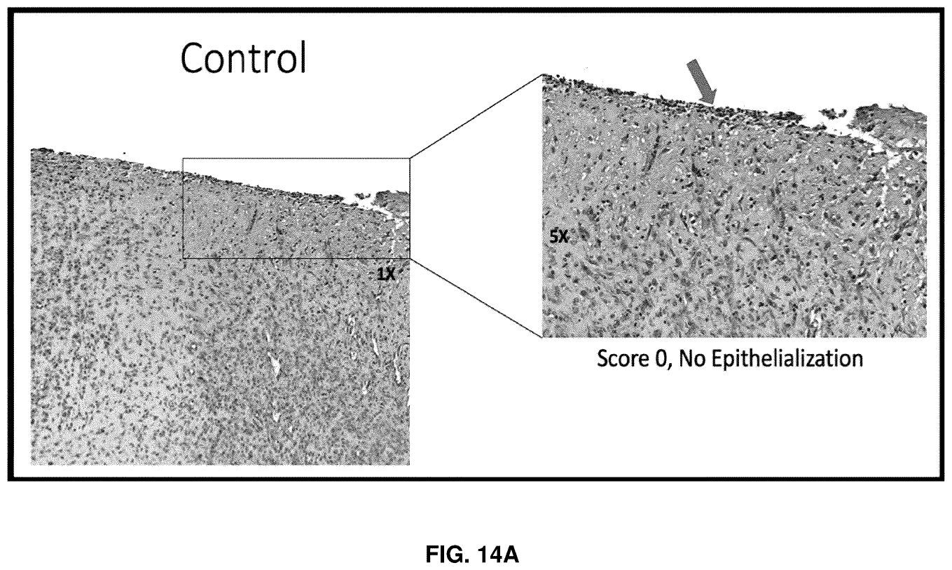

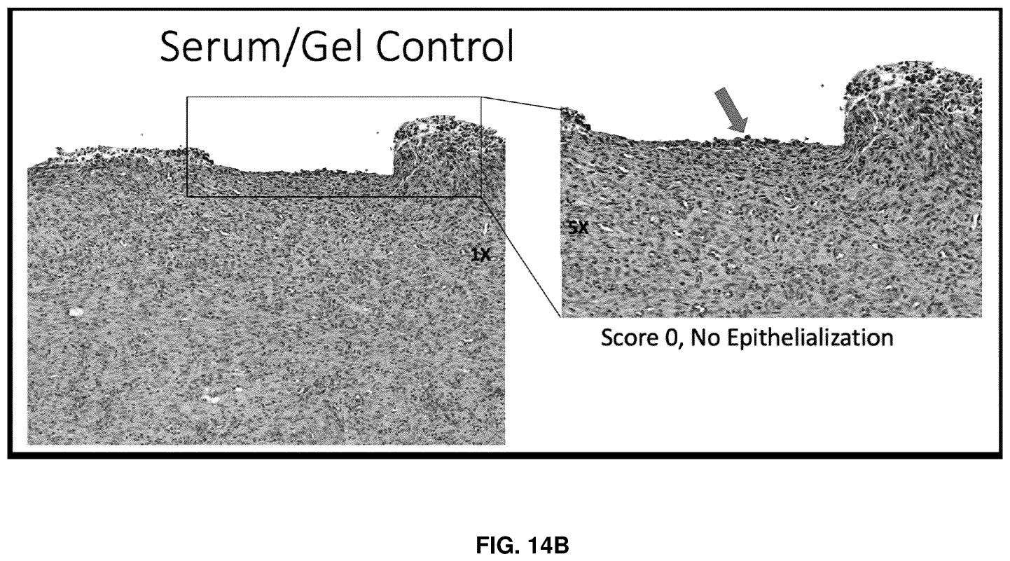

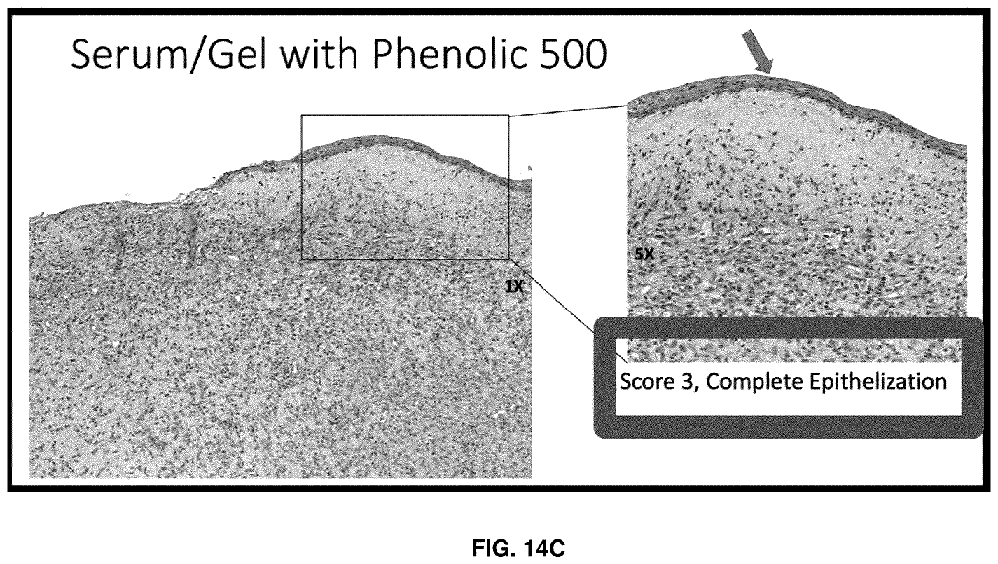

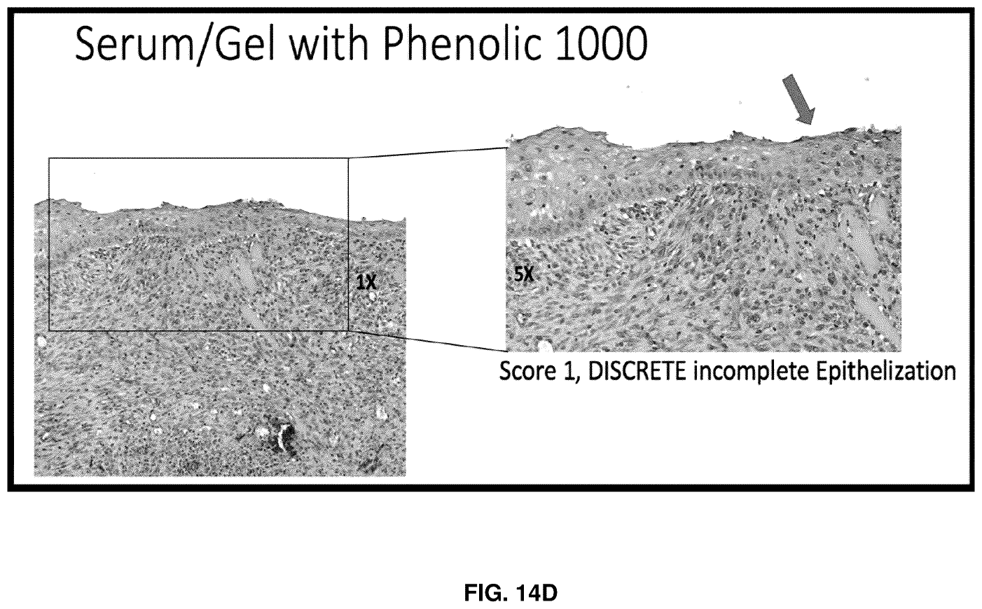

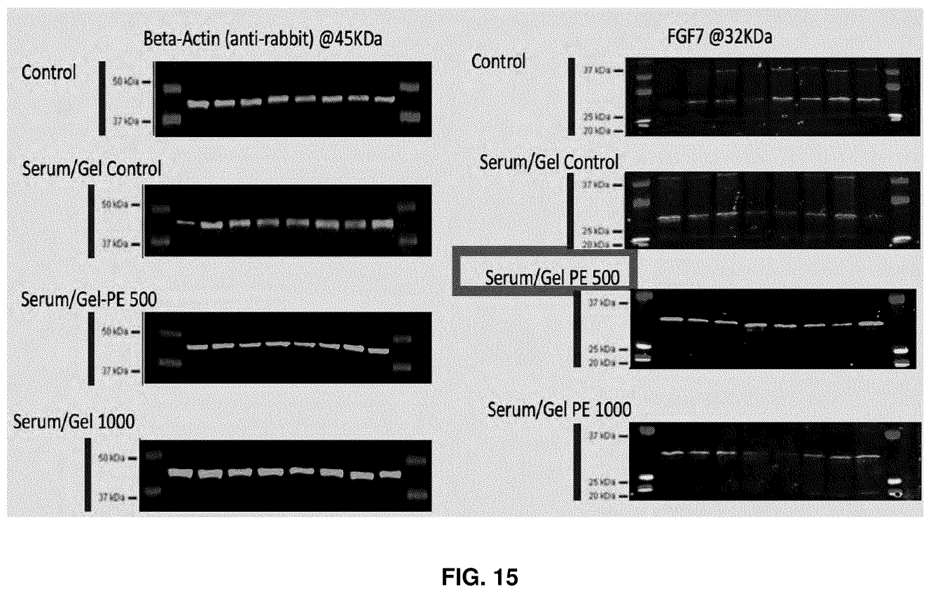

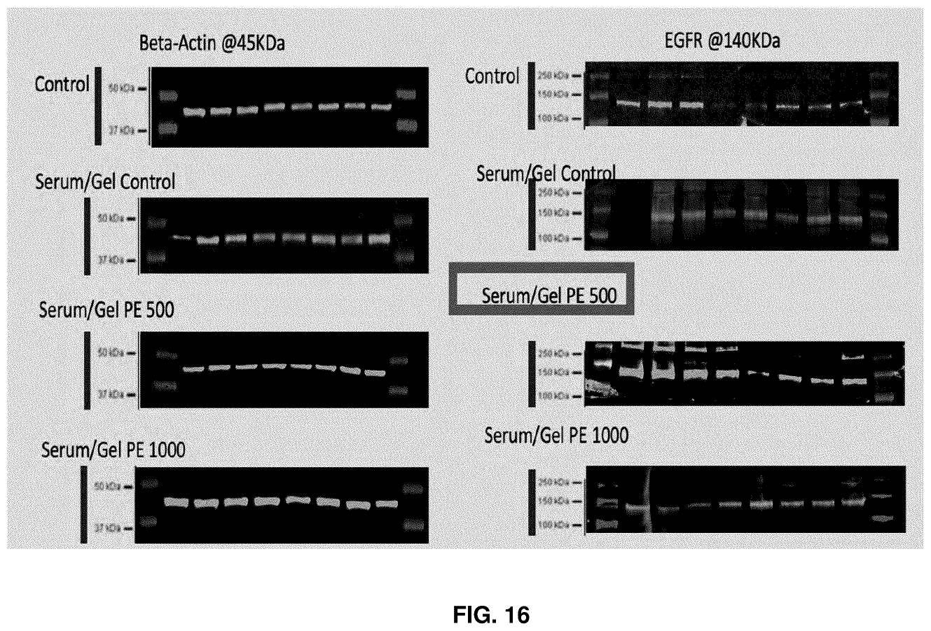

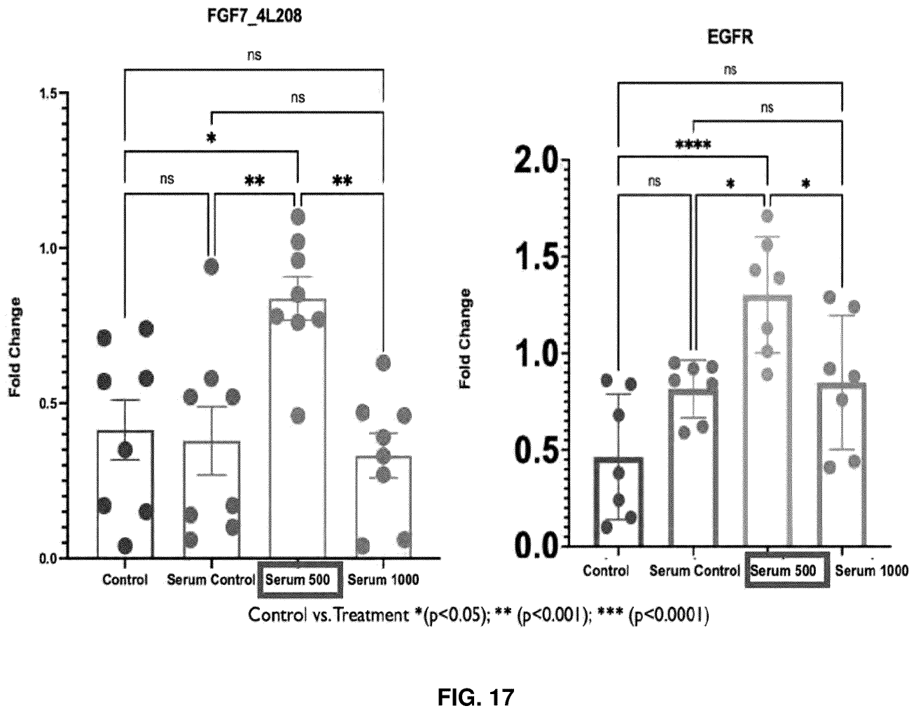

FIGS. 14A-14D show the re-epithelialization score results from the serum/gel. FIGS. 15-17 show the results of the re-epithelialization protein analysis of FGF7 and EGFR from the serum/gel. Phenolic extract from wild blueberries in the serum/gel formulations upregulates EGFR and FGF7 proteins for epidermal skin regeneration during wound re-epithelialization.

This example shows that phenolic acids from wild blueberries in serum/gel at 500 μg/ml, when compared to a control, possess vascular remodeling ability via (1) a 12% increase in wound closure, as shown in FIG. 7, (2) a 20% increase in cellular vascularization on wound area, as shown in FIGS. 8-9, (3) an organized pattern of collagen deposition on wound area, as shown in FIGS. 10, and (4) a highly significant increase in vascular (VEGFA p value <0.0001) and collagen (Col1a1, p value <0.0058) proteins, as shown in FIGS. 11-13. This example also demonstrates that phenolic acids from wild blueberries in serum/gel at 500 μg/ml when compared with control result in complete re-epithelialization and epidermal regeneration of wound area as shown in FIG. 14A-14D, and a significant increase in fibroblasts (FGF7 p value <0.05) and epidermal (EGFR, p value <0.0001) proteins as shown in FIGS. 15-17 for epidermal regeneration during wound re-epithelialization.

In sum, this example shows significant effects of phenolic extracts from wild blueberry as a natural, inexpensive wound healing product, and demonstrates its usefulness as a treatment for patients with acute and chronic wounds. Different concentrations of phenolic acid extracts delivered by two vehicles, namely, a gel/serum and a cream, were evaluated. The serum/gel composition delivering the phenolic fraction at P500 μg/ml significantly promoted wound closure by 12% above the control (control=nothing on the wound). The phenolic fraction (P500 μg/ml) showed a greater ability to be highly vascularized compared to the control and the other phenolic concentrations, and resulted in a 20% increase in cellular vascularization on the wound area. Immune cell activity significantly decreased with both serum/gel P500 μg/ml and P1000 μg/ml. Complete re-epithelialization occurred with serum/gel P500 μg/ml compared to other treatments. Serum/gel P500 μg/ml treatment promoted dense and thick deposition of collagen and adequate tissue remodeling compared to the other treatments. Proteins on the wound associated with vascularization, collagen formation, cell growth, tissue repair, and anti-inflammation were significantly increased with treatment of the wound with serum P500 μg/ml. Notably, the cream formulation did not show significant results in the speed of wound closure, indicating that not just any vehicle or carrier for the phenolic acid extract results in a formulation which is effective at promoting wound healing.

### Example III—CNF Hydrogel for Delivery of Wild Blueberry Extract

Wild blueberries (WBs) contain high concentrations of bioactive compounds including phenolic acids and anthocyanins, which have been shown to play a significant role in cell migration. Physiological phenomena such as angiogenesis and wound healing may be promoted through controlled application of the wild blueberry extract.

Hydrogels are useful for drug delivery and wound healing. TEMPO-oxidized CNF hydrogels are both biocompatible and modifiable. TEMPO-oxidized CNF can be crosslinked using citric acid, which can also be used to prevent oxidation of the WB extract. This example describes testing a homogeneous TEMPO-oxidized CNF hydrogel with Coomassie brilliant blue (CBB) as an indicator dye and model for a WB extract in the loaded TEMPO-oxidized CNF hydrogel. As demonstrated in this example, the TEMPO-oxidized CNF hydrogel is capable of controllably releasing the CBB.

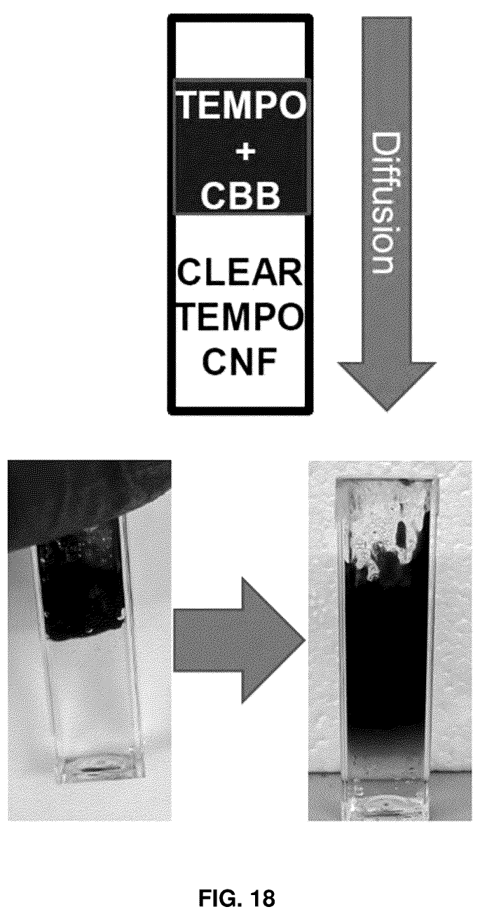

A 1.1 wt % TEMPO-oxidized CNF was used for the top and bottom hydrogel layers. In order to load the TEMPO-oxidized CNF into the cuvette, a modified syringe was used. The clear bottom layer of TEMPO-oxidized CNF was then degassed in a sonicating bath to ensure homogeneity. Four separate hydrogels were loaded with CBB ranging in concentration from 5 mM to 50 mM. The dyed TEMPO-oxidized CNF was then layered carefully on top of the clear TEMPO-oxidized CNF using another syringe. (FIG. 18.) The cuvette was then immediately covered and placed into the spectrometer for analysis.

The optimal spectrometer settings were determined. An optical cable was selected based on the comparison of four sizes ranging from 300 μm to 1500 μm. The 600 μm cable produced the most consistent results across all concentrations and light intensity levels. The medium light intensity was found to produce the most balanced results in combination with the 600 μm optical cable.

The diffusion of CBB through the TEMPO-oxidized CNF hydrogel is shown in FIG. 19. Four concentrations of TEMPO-oxidized CNF+CBB were examined using the spectrometer: 5 mM, 15 mM, 30 mM, and 50 mM. The absorbance versus time plot results in a curve that first appears exponential before approaching an absorbance upper threshold. As the CBB concentration of the loaded TEMPO-oxidized CNF increased, the time to reach the absorbance upper threshold decreased.

A WB extract can be used in the loaded top layer of the TEMPO-oxidized CNF hydrogel in place of the CBB. Citric acid can be used to crosslink the hydrogel. The weight percent of TEMPO-oxidized CNF can be raised through vacuum filtration.

### Methodology

To determine the optimal settings of the spectrophotometer, an experiment was

designed to examine four variables: CBB concentration, optical cable diameter, fixed light intensity, and integration time. First, seven concentrations of CBB and water were created at 0.00001 mM, 0.0001 mM, 0.001 mM, 0.01 mM, 0.1 mM, 1.0 mM, and 1 mM. These concentrations were loaded into cuvettes to be examined using the spectrophotometer and capped with parafilm to prevent evaporation. To connect the light collector to the detector an optical cable was used. Four optical cable diameters were tested in this experiment: 300 μm, 600 μm, 1000 μm, and 1500 μm. Another variable examined was the fixed LED intensity at the settings of low, medium, and high. Finally, two integration times were tested at 1 and 2 seconds. Five spectra were gathered using all the possible combinations of these variables. A total of 840 spectra were recorded to be used for later analysis.

Following the optimization of the spectrophotometer, settings were selected to observe the CBB diffusion experiment through the hydrogels. An optical cable of 600 μm was used with medium light intensity and an integration time of 1 second. These spectrophotometer settings were directly controlled using LabVIEW.

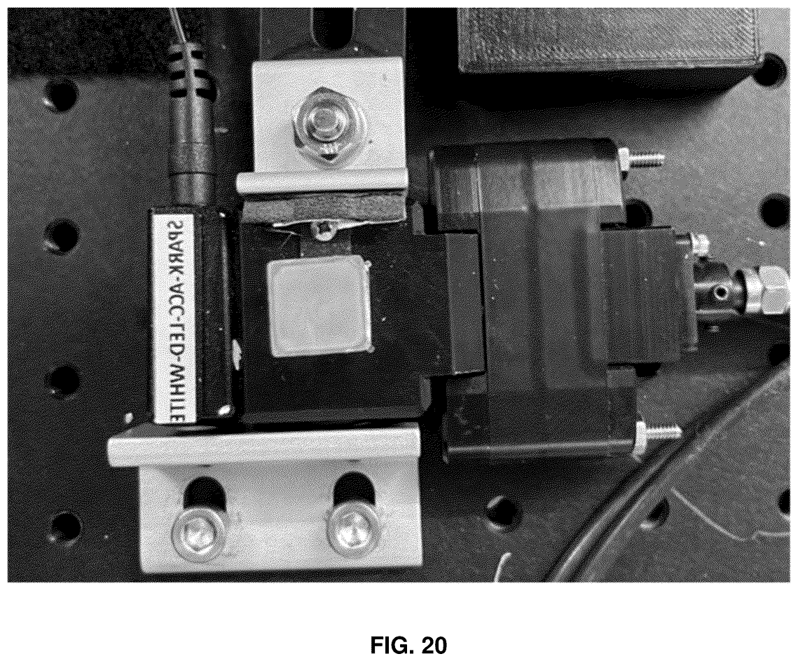

The TEMPO-oxidized CNF hydrogel used in this example was provided at a weight percentage of 1.1%. This unmodified 1.1% hydrogel was used as the skin tissue model for this experiment. To load this gel into the bottom of the clear cuvette, a 1 ml syringe was used. It is important to aspirate and dispense the hydrogel carefully to eliminate air pockets. Once 2 ml of the gel was carefully dispensed in the bottom of the cuvette, a small stirrer was used to remove any large remaining air pockets. The cuvette containing the gel was then taken and partially submerged in a sonicating water bath to remove any remaining air. After 15-20 min of degassing, the cuvette was removed and inspected for remaining air pockets. On some occasions, further careful manipulation may be required with the stirrer to create a homogeneous hydrogel. Removal of all air is important as it can heavily impact the diffusion of the CBB into the gel. In separate beakers, unmodified 1.1% TEMPO-oxidized CNF was mixed with CBB to reach the desired concentrations of 5 mM, 15 mM, 30 mM, and 50 mM. The CBB-loaded hydrogel can then be loaded on top of the 2 ml of unmodified hydrogel. The layer of loaded gel should be a volume of 1 ml. Another 1 ml syringe was used to extract the loaded gel from the beaker and dispense it carefully on top of the clear hydrogel. Once the CBB gel was layered in the cuvette with minimal air pockets, the cuvette was immediately placed into the center of the spectrophotometer as shown in FIG. 20. The cuvette was then covered by a light-blocking cap, and spectra were taken every ten minutes for up to 35 hours. The LabView program then saved all the data to a text file to be analyzed later.

### Results

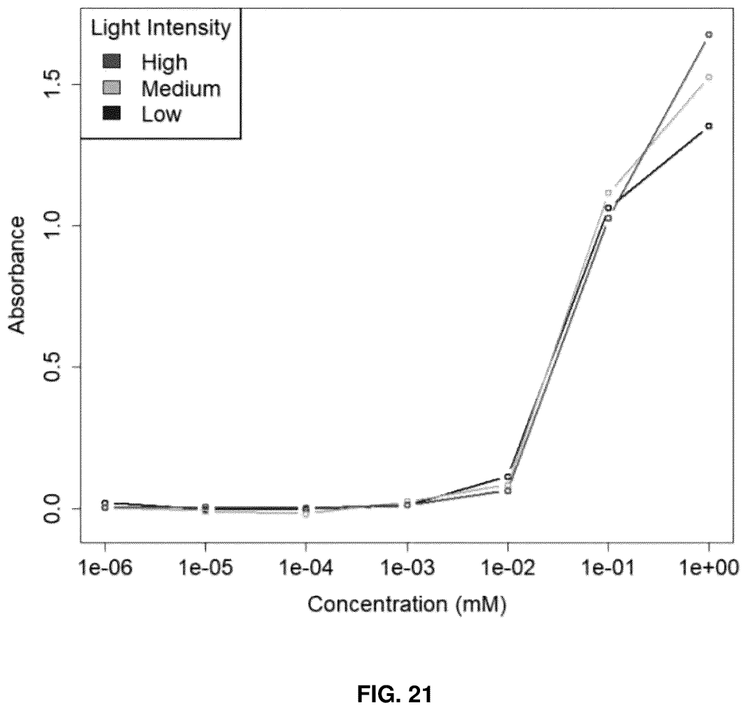

After completing the experiment to optimize the spectrophotometer settings, the data was graphed as seen in FIG. 21, which plots the absorbance vs the seven concentrations of CBB used in the experiment. Each line of the graph represents a different light intensity setting. In this case, raising the light intensity level resulted in a slight increase in absorbance values. Because of this variability between light setting absorbance values at the higher concentrations, a medium light setting was selected. It was also determined from FIG. 21 that the concentration of CBB loaded into the hydrogel should be greater than 0.1 mM as CBB solutions below this concentration did not result in a significant reading of absorbance values. For each optical cable diameter and integration time, a graph in the same format as FIG. 21 was generated. From these other charts, the optimal optical cable diameter was determined to be 600 μm. The larger optical cable diameters overloaded the detector resulting in a less reliable signal than the 600 μm cable. The integration time selected was 1-second. The 2-second integration time resulted in higher absorbance variability and would occasionally result in exceeding the reading capabilities of the spectrophotometer. The final settings selected for the CBB hydrogel diffusion testing were medium light, 600 μm optical cable diameter, and 1-second integration time.

Using these spectrophotometer settings, four concentrations of CCB-loaded hydrogels were observed diffusing through the clear hydrogel over time. FIG. 19 shows the absorbance of each of these loaded concentrations as they diffuse over time through the gel. The 15 mM, 30 mM, and 50 mM gels all diffused slowly initially, before accelerating and then leveling off at an absorbance value close to 1.2. The 5 mM gel was a much lower initial concentration compared to the other gels, and after 35 hours, continued to diffuse through the clear hydrogel at a steadier rate.

### Example IV—Wild Blueberry Extract with CNF Hydrogel Promotes Wound Healing

As shown in the above examples, phenolic extracts derived from wild blueberries have the ability to significantly promote wound healing and increase tissue repair, revascularization, re-epithelialization, and collagen formation. This example describes using a TEMPO-oxidized CNF hydrogel as a carrier for the phenolic acid extract. The carrier preserves and enhances the effect of the extract for wound healing without altering the effect of the wild blueberry extract on the wound.

In pre-clinical studies with animals with excision wounds, the wild blueberry extract (phenolic fraction (P500 μg/ml)-citric acid crosslinked t-CNF hydrogel formulation significantly promoted wound closure by 27.5% above control (nothing on the wound) and 41.7% above Neosporin (antibiotic most commonly used for wound healing). The formulation also exhibited higher re-epithelialization and collagen formation.

Crosslinking chemistry does not require the use of non-biocompatible chemical crosslinking agents. The same cross-linking agent (citric acid) is an effective preservative and even adjuvant for the wild blueberry extract, enhancing wound healing efficacy relative to control and Neosporin. Additionally, citric acid does not have any effect on all parameters studied.

### Materials and Methods

### Blueberry Extract Nanogel

A phenolic acid extract from wild blueberry (V. angustifolium) powder was obtained as described in U.S. Application Publication No. 2020/0330541 A1. An HPLC profile of the phenolic acid extract is shown in FIG. 1. The phenolic acid extract included mainly chlorogenic acid (10.23±1.8 mg/mL) with traces of ferulic and caffeic acids. The concentration of the phenolic acid was measured and the extract was diluted to 100 μg/ml.

TEMPO-oxidized CNF was obtained. To make a concentration of 500 μg/ml phenolic acid in CNF hydrogel, 5 mLs of 1000 μg/ml phenolic acid in Pierce water was added to LC-MS Grade Tincture 9 grams (10,000,000 μg) Tempo-oxidized CNF along with 1 gram of citric acid. The mixture was stirred for about 1-2 minutes until properly mixed with a consistent appearance. The desired volume of mixture was added into a container to form the desired shape, and then placed in 4° C. for 24 hours and covered to maintain moisture. To form the proper shape, the mixture was left uncovered for 40 hours at 4° C.

### Preparation of Blueberry Extract

Blueberry extract was removed from −80° F. and placed into a refrigerator overnight to allow to thaw completely.

### Preparation of 3.6% TEMPO-Oxidized CNF

163.6364 grams of 1.1% TEMPO-oxidized CNF (TCNF) solution were weighed out and added to a vacuum filter with 0.45 μm filter paper. Vacuum filtration was begun and continued until the TCNF solution reached a weight percentage of 3.6%. Once the TCNF reached 3.6%, the solution was removed from vacuum filtration and placed into a small beaker and covered with parafilm before placing it into the refrigerator.

### Preparation of 1500 μM Citric Acid Solution

0.02304 grams of citric acid were weighed out. 80 mL of DI water were added to the citric acid and the solution was mixed until completely dissolved. The citric acid solution was covered with parafilm and placed into the refrigerator along with the prepared TCNF.

### Forming Blueberry Extract Gel

The blueberry extract gel was prepared weekly. 4 grams of 3.6% TCNF solution were removed from the refrigerator and placed into a small beaker. 6 mL of 1000 μm/mL phenolic acid extract were added to the small beaker and the mixture was mixed thoroughly until completely combined. 2 mL of the citric acid solution were added to the TCNF blueberry extract solution and mixed immediately for three minutes to ensure the citric acid was completely mixed with the extract-TCNF solution. This produced a 12 mL solution of 1.2% TEMPO, 500 μm/mL extract, and 250 μM citric acid.

### Forming Control Gel

The control gel was prepared weekly. 4 grams of 3.6% TCNF solution was removed from the refrigerator and placed into a small beaker. 6 mL of DI water were added to the small beaker and mixed thoroughly until completely combined. 2 mL of citric acid solution were added to the TCNF and mixed immediately for three minutes to ensure the citric acid was completely mixed with the TCNF solution.

### Results

FIG. 22 shows the results. As seen in FIG. 22, the nanogel phenolics increased the percentage wound closure with 27.5% more than control and 41.7% more than Neosporin. Nanogel phenolics had a significant increase in the rate of wound closure compared to other treatment groups (0.0001 for control and Neosporin, 0.0002 for nanogel control). Notably, the nanogel phenolics exhibited better wound closure than not only a negative control (nothing on the wound) but also a TCNF nanogel control without the extract. In fact, the wound closure was significantly better than that observed with the TCNF nanogel control. When this result is considered in combination with the results described in Example II above, where other formulations of the phenolic acid extract were tested in wound healing assays, it is clear that the combination of the phenolic acid extract with the TCNF hydrogel provides improved wound healing better than phenolic acid alone, better than TCNF hydrogel alone, better than the phenolic acid extract in a serum/gel or cream formulation, and better than what would be expected from a merely additive benefit from combining TCNF hydrogel with the phenolic acid extract.

The description of the various aspects and embodiments of the present invention has been presented for purposes of illustration and description. It is not intended to be exhaustive of all embodiments or to limit the invention to the specific aspects disclosed. Obvious modifications or variations are possible in light of the above teachings and such modifications and variations may well fall within the scope of the invention as determined by the appended claims when interpreted in accordance with the breadth to which they are fairly, legally and equitably entitled.

What is claimed is:

1. A method of promoting wound healing in a subject, the method comprising administering an effective amount of a composition to a subject in need of wound healing to promote wound healing in the subject, wherein the composition comprises a nanocellulose hydrogel and an extract of berries from the Vaccinium family.

2. The method of claim 1, wherein the berries are blueberries.

3. The method of claim 1, wherein the extract is a phenolic-rich fraction.

4. The method of claim 3, wherein the phenolic-rich fraction comprises chlorogenic acid.

5. The method of claim 3, wherein the phenolic-rich fraction comprises ferulic acid and caffeic acid.

6. The method of claim 1, wherein the nanocellulose hydrogel comprises a TEMPO-oxidized CNF hydrogel.

7. The method of claim 6, wherein the TEMPO-oxidized CNF hydrogel is crosslinked with citric acid.

8. A composition comprising:
an extract of berries from the Vaccinium family; and
a nanocellulose hydrogel.

9. The composition of claim 8, wherein the extract comprises phenolic acids.

10. The composition of claim 8, wherein the phenolic acids include chlorogenic acid.

11. The composition of claim 10, wherein the phenolic acids further include ferulic acid and caffeic acid.

12. The composition of claim 8, wherein the nanocellulose hydrogel comprises a TEMPO-oxidized CNF hydrogel.

13. The composition of claim 12, wherein the TEMPO-oxidized CNF hydrogel is crosslinked with citric acid.

14. A composition comprising:
a phenolic acid extract of berries from the Vaccinium family;
phenethyl alcohol;
hydrolyzed hyaluronic acid; and
sodium hyaluronate;
wherein the composition is in the form of a serum or gel.

15. The composition of claim 14, wherein the phenolic acid extract comprises chlorogenic acid, ferulic acid, and caffeic acid.

16. A method of promoting wound healing, the method comprising administering an effective amount of the composition of claim 14 to a subject in need of wound healing to promote wound healing in the subject.

17. A composition comprising:
a phenolic acid extract of berries from the Vaccinium family;
shea butter glycerides;
phenethyl alcohol;
castor seed oils;
cetearyl alcohol;
glyceryl stearate;
sodium stearoyl lactylate; and
balsam peru oil;
wherein the composition is in the form of a cream.

18. The composition of claim 17, wherein the phenolic acid extract comprises chlorogenic acid, ferulic acid, and caffeic acid.

19. A method of promoting wound healing, the method comprising administering an effective amount of the composition of claim 17 to a subject in need of wound healing to promote wound healing in the subject.
